(19) United States
(12) Patent Application Publication
(10) Pub. No.: US 2026/0053568 A1
(43) Pub. Date: Feb. 26, 2026

(54) MARKERLESS TRACKING AND LATENCY REDUCTION APPROACHES, AND RELATED DEVICES

(71) Applicant: Monogram Technologies Inc., Austin, TX (US)

(72) Inventors: Kamran Shamaei, Austin, TX (US); Pedro Alfonso Patlan Rosales, Buda, TX (US); Hrisheekesh Patil, Waltham, MA (US); Sanath Vurelli, Waltham, MA (US); Benjamin Sexson, Austin, TX (US); Juhyun Son, Houston, TX (US)

(73) Assignee: Monogram Technologies Inc., Austin, TX (US)

(21) Appl. No.: 19/368,500

(22) Filed: Oct. 24, 2025

Related U.S. Application Data
(63) Continuation of application No. PCT/US2024/026534, filed on Apr. 26, 2024.
(60) Provisional application No. 63/498,504, filed on Apr. 26, 2023.
(60) Provisional application No. 63/501,022, filed on May 9, 2023.
(60) Provisional application No. 63/504,285, filed on May 25, 2023.
(60) Provisional application No. 63/569,471, filed on Mar. 25, 2024.

(51) Int. Cl. A61B 34/10; A61B 34/00; A61B 34/20; A61B 34/30

(52) U.S. Cl. CPC A61B 34/10; A61B 34/20; A61B 34/25; A61B 34/30; A61B 2034/105; A61B 2034/2046

(57) ABSTRACT

A method for modeling patient anatomy includes, generating and segmenting a point cloud of the patient anatomy to identify visible bone surface and soft tissue surface regions of the patient anatomy, registering a reference bone model to the identified visible bone surface regions to provide an initial patient anatomy model having an estimation of a pose of bone portions of the patient anatomy, augmenting the initial patient anatomy model with soft tissue bodies based on the estimation of the pose of the bone portions, where the augmenting provides a full patient anatomy model having (i) the soft tissue bodies representing soft tissue portions of the patient anatomy and (ii) elements representing the bone portions of the patient anatomy, and registering the full patient anatomy model to the segmented point cloud such that the full patient anatomy model accurately reflects a current position and pose of the patient anatomy.

### CROSS-REFERENCE TO RELATED APPLICATIONS

This application is a continuation of International Application Number PCT/US2024/026534, entitled “MARKERLESS TRACKING AND LATENCY REDUCTION APPROACHES, AND RELATED DEVICES”, filed Apr. 26, 2024, and published Oct. 31, 2024 as International Publication Number WO 2024/226989 which is hereby incorporated by reference in its entirety, and which claims the benefit of U.S. Provisional Application Numbers 63/498,504 filed Apr. 26, 2023 and entitled “MARKERLESS TRACKING”, 63/501,022 filed May 9, 2023 and entitled “MARKERLESS TRACKING APPROACHES AND RELATED DEVICES”, 63/504,285 filed May 25, 2023 entitled “MARKERLESS TRACKING AND LATENCY REDUCTION APPROACHES, AND RELATED DEVICES”, and 63/569,471 filed Mar. 25, 2024 entitled “MARKERLESS TRACKING AND LATENCY REDUCTION APPROACHES, AND RELATED DEVICES”.

### BACKGROUND

In various clinical applications it may be beneficial to relay to a processor information about the current and/or past locations and orientations (special position or pose) of an object, i.e., to track the object. Frequently, two-dimensional (2D) and/or three-dimensional (3D) graphical representations of tracked objects are displayed to a user on a monitor/display/screen, for instance. In some examples, they are displayed using augmented reality to overlay graphical element(s) over a video/live view to an environment. By way of nonlimiting example, pre-operatively captured data, such as reconstructed computed tomography (CT) views of a patient's pre-operative anatomy, may be displayed by the tracking system. A tracking system that displays views of the anatomy and/or surgical instruments is sometimes referred to as a navigation system, and a navigation system may display generalized anatomical models.

### SUMMARY

Shortcomings of the prior art are overcome and additional advantages are provided.

In one example, a computer-implemented method is provided for tracking at least one object in an environment. The method includes generating a point cloud from image data obtained from one or more cameras. The generating the point cloud includes imaging, using the one or more cameras, the at least one object, where each camera of the one or more cameras provides a respective image stream of one or more image streams. The generating the point cloud also includes generating the point cloud as a three-dimensional (3D) representation of the at least one object based on the one or more image streams. The method additional includes segmenting the generated point cloud into a segmented object model for the at least one object, where the segmenting identifies the at least one object and extremities of the at least one object as exhibited by the generated point cloud. The method also includes deforming a predefined mesh model, representative of at least a portion of the at least one object, to correlate to the segmented object model, the deforming providing a position of the at least one object in the environment.

In another example, a computer-implemented method for modeling patient anatomy in a current position and pose, including registering bone that is obscured by soft tissue, is provided. The method includes generating a point cloud of the patient anatomy. The method also includes segmenting the point cloud to identify visible bone surface regions of the patient anatomy and visible soft tissue surface regions of the patient anatomy. The method additionally includes registering a reference bone model for the patient to the identified visible bone surface regions, where the registering provides an initial patient anatomy model having an estimation of a pose of bone portions of the patient anatomy. The method further includes augmenting the initial patient anatomy model with soft tissue bodies, each comprising a respective volume and respective surface, based on the estimation of the pose of the bone portions of the patient anatomy, where the augmenting provides a full patient anatomy model having (i) the soft tissue bodies representing soft tissue portions of the patient anatomy and (ii) elements representing the bone portions of the patient anatomy. The method also includes registering the full patient anatomy model to the segmented point cloud such that the full patient anatomy model accurately reflects a current position and pose of the patient anatomy.

In yet another example, a computer-implemented method for robotic surgery is provided. The method includes one or more of navigating or controlling a robot during a navigated robotic surgery based on markerless tracking of at least one object.

Additional aspects of the present disclosure are directed to systems and computer program products configured to perform the methods described above and herein. The present summary is not intended to illustrate each aspect of, every implementation of, and/or every embodiment of the present disclosure. Additional features and advantages are realized through the concepts described herein.

### BRIEF DESCRIPTION OF THE DRAWINGS

Aspects described herein are particularly pointed out and distinctly claimed as examples in the claims at the conclusion of the specification. The foregoing and other objects, features, and advantages of the disclosure are apparent from the following detailed description taken in conjunction with the accompanying drawings in which:

FIG. 1A depicts an example in which tracking arrays are attached to anatomy for tracking the anatomy;

FIG. 1B depicts an example of array articulation via a series of joints;

FIG. 2 depicts an example beacon for radar-based bone tracking;

FIG. 3 depicts an example in which cartilage obstructs the view to bone surface underneath the cartilage;

FIGS. 4A-4G present an example depiction of anatomy pos tracking, in accordance with aspects described herein;

FIG. 5 depicts an example of image cropping;

FIGS. 6A-6F present another example depiction of anatomy pose tracking, in accordance with aspects described herein;

FIG. 7 depicts an example in which color-based filtering is used to identify anatomy of interest by filtering an image, in accordance with aspects described herein;

FIGS. 8A-8B depict an example in which color-based filtering is used to filter an image, in accordance with aspects described herein;

FIG. 9 depicts an example presentation of a high-density point cloud of patient anatomy, in accordance with aspects described herein;

FIG. 10 depicts an example in which multiple cameras are positioned around the operating theater to capture multiple views of anatomy of interest, in accordance with aspects described herein;

FIG. 11 depicts an example camera with depth sensing capability;

FIGS. 12A-12B depict example environments employing edge processing to facilitate anatomical tracking, in accordance with aspects described herein;

FIG. 13A-13B depict example differentiated structured light camera projection approaches, in accordance with aspects described herein;

FIG. 14 depicts an example of fixing objects of interest for markerless tracking, in accordance with aspects described herein;

FIGS. 15A-15F illustrate additional examples of fixing objects to facilitate markerless tracking in accordance with aspects described herein;

FIGS. 16A-6B depict example external fixation pins also serving as tracking arrays, in accordance with aspects described herein;

FIG. 17 depicts an example exposed knee joint and anatomical features thereof with a highlighted anatomical region in accordance with aspects described herein;

FIG. 18 depicts an example computer system to incorporate and/or use aspects described herein.

### DETAILED DESCRIPTION

Described herein are aspects (e.g., methods, systems, computer program products) for tracking objects, for instance bone(s) and/or other anatomy. In some examples, the objects are tracked directly and without/absent relying on/placing known objects in a scene (for example rigid markers for tracking in a surgical operation) or on pre-operative patient imaging, for instance. Also described are methods for mitigating latency, which in some embodiments can be used in conjunction with markerless tracking to facilitate such processes.

In examples, markerless tracking may be accomplished using any of varying types of cameras/sensors, for instance Red, Green, Blue wavelength (RGB) and/or RGBD (red, blue, green depth) camera/sensor(s) as described herein. Though RGBD technology is mentioned in examples used herein, various other technologies may be used to provide depth information in conjunction with image data. For instance, structured-light camera(s) (sometimes referred to as “structured-light 3D scanners”, “depth cameras”, or “3D cameras” but referred to herein as an example of a “camera” for convenience) may be used to measure the 3D shape of an object, for example by capturing 3D information about a scene or an object by projecting a known pattern of light onto the target and analyzing its deformation. Often times, such cameras/scanners include a light source and camera sensor. In one example, the light source projects light as a series of parallel patterns onto a scan target. The patterns become distorted when the light projects onto the object's surface, and the associated camera produces/captures images of this distortion and sends them to 3D scanning software for processing. Other devices providing depth-sensing and/or depth information indicating distance to an imaged target may be used.

A more detailed example of how a structured light camera might work is provided as follows:
Pattern Projection: The structured light camera emits a structured pattern of light onto the scene or object. This pattern can be a grid, a series of lines, or a random pattern, as examples. The projected pattern is usually created using a laser or a light source combined with a diffuser and a lens system to project the pattern clearly onto the target;
Deformation Analysis: The structured light pattern interacts with the surfaces of the target/object in the scene. The shape, contours, and depth discontinuities of the object cause the pattern to deform and create distortions in the captured image;
Image Capture: The camera, usually equipped with an image sensor such as a CMOS or CCD sensor, captures the deformed pattern by imaging the scene. The camera is to be synchronized with the projected pattern to ensure proper capture;
Data Processing: The captured image is then processed to extract depth information. This involves analyzing the deformations and distortions in the pattern caused by the object's shape. Typically, image processing techniques such as triangulation, stereo matching, or phase-shift analysis are employed to determine the depth of each point in the scene;
Depth Calculation: Using the extracted information, the camera calculates the distance from the camera to each point in the scene or object. By analyzing the disparities between corresponding points in the projected pattern and the captured image, the camera can calculate the depth values;
Point Cloud Generation: The structured light camera generates a point cloud, which is a set of three-dimensional coordinates representing the shape of the object or the scene. Each point in the cloud corresponds to a specific location in space and has associated depth information;
Applications: The generated point cloud data can be used for various applications, such as 3D scanning, object recognition, augmented reality, virtual reality, robotics, and more.

It is important to note that different structured light cameras may use slightly different techniques or variations of the process described above. However, the fundamental principle remains the projection of a known pattern onto the target and analyzing the deformations of the pattern, as imaged, to extract depth information.

By way of nonlimiting example, the majority of existing navigation systems used in surgical activities either 1) track objects by proxy, i.e., they track the position of objects by rigidly affixing arrays that help facilitate tracking the object of interest, or 2) track objects with such high latency that the real-time position of the objects is unknown (i.e., a display of the position at a given point in time lags relatively significantly behind the actual position of the object at that point in time). An example of this is FLASH registration technology from 7D Surgical Inc. In contrast, aspects described herein provide methods and approaches that provide low-latency (for instance, less than 20 milliseconds at 60 Hz) markerless tracking.

For many navigated surgical procedures, tracking arrays are used to track the position of objects of interest. Tracking arrays are often rigidly mounted to the anatomy of interest, for example to a bone. In many current approaches, the arrays are rigidly fixed with 3 millimeter (mm) bicortical bone pins that are generally 50 mm long and stiffened via a sleeve between the pins. These arrays are known objects that in some embodiments may include distinct features, such as retroreflective markers, that facilitate tracking with specialized cameras. It is imperative under these approaches that the array be rigid relative to the anatomy of interest, and this configuration may not provide stability of the array in all directions.

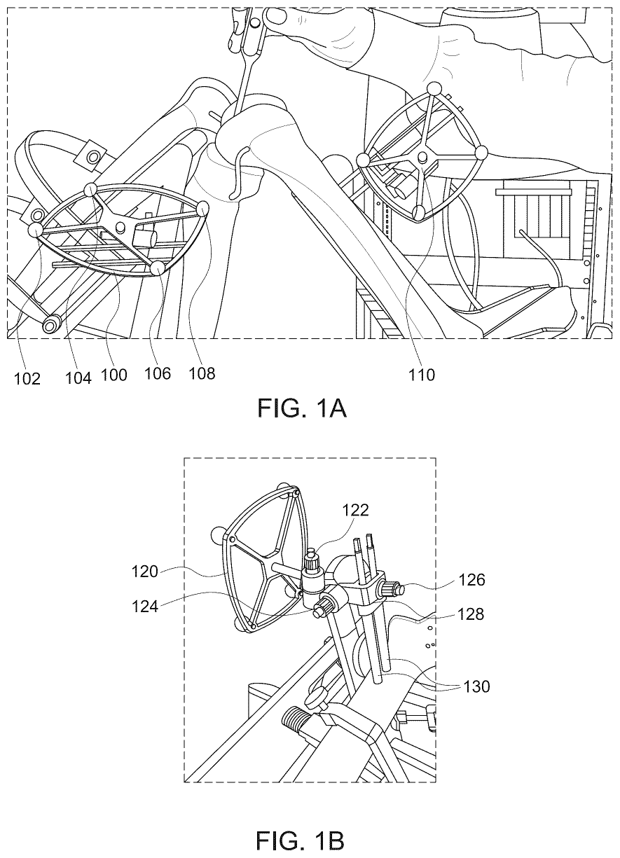

Referring to FIG. 1A, which depicts an example in which tracking arrays are attached to anatomy for tracking the anatomy, to track objects of interest using tracker array(s) requires line-of-sight between the tracker array(s) 100, 110 and the localization camera(s) (not pictured). The array fiducials (e.g., 102, 104, 106, 108 using array 100 as an example) also need to be generally oriented such that all of them are visible to the camera(s). This requires full articulation of the array relative to the mount. Conventional methods achieve this with a series of joints tightened with instrumentation. Referring to FIG. 1B, which depicts an example of array articulation via a series of joints, articulation/movement of array 120 is provided by three points of articulation—122, 124, 126—each adjusted by loosening a bolt, making the adjustment, then tightening the bolt. Points 122 and 124 provide rotatable joints, while point 126 enables the sliding member 128 to slide along the pins 130 to move the array assembly nearer-to, or farther from, the anatomy. In this setup, the mechanism lacks rigidity until all of the joints are tightened. This approach therefore requires some dexterity and two hands of a user to orient and tighten, which adds time, frustration, and cost.

In these setups, the arrays must be fixated to the bone, and their position relative to the bone must remain unchanged after registration. To accurately track an object, it is also frequently helpful to have a preoperative image of that object, such as a CT scan. The cost to obtain a CT scan can be expensive and time consuming. While aspects described herein could work with such a preoperative image if available, it may not be required, i.e., aspects described herein may not need such a preoperative image (e.g., CT scan).

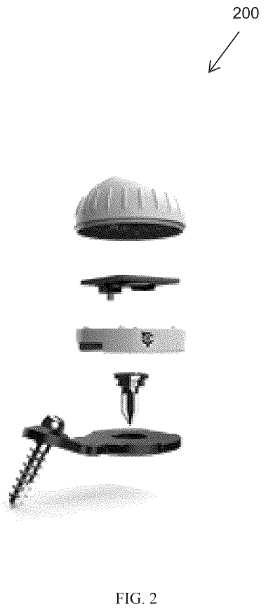

The aforementioned example describes the placement of arrays for tracking object position optically. There may be other methods of tracking that require the placement of a fixed marker on the object of interest. By way of nonlimiting example, an approach exists that proposes rigidly placing a beacon for radar-based bone tracking. FIG. 2 depicts such an example beacon 200 for radar-based bone tracking. In contrast, aspects described herein can eliminate the need for placement of any tracking markers, since aspects described herein provide approaches for markerless tracking, that is, tracking without use or reliance on markers. By way of nonlimiting example, placing fixed markers introduces surgical time, surgical complexity, and surgical cost. Bone pins are also invasive and are often inserted outside of the incision.

Another advantage of markerless tracking is that it avoids having to work around markers. For instance, the human and/or robot does not have to worry about interference or other undesirable interaction with a marker when performing a surgery. By way of specific example, the human/robot when cutting does not have to worry about cutting into a bone pin or other marker being used to facilitate tracking.

In accordance with some aspects described herein, novel approaches, processes, and methods of tracking objects, for example anatomy and optionally other objects such as surgical instruments, are provided that do not rely on the placement of known objects into a scene. Aspects presented herein can, as examples, reduce surgical setup time, reduce the number of surgical steps required to conduct any navigated procedure, minimize the need to alter the anatomy and associated risks from placing tracking arrays, minimize the risk of tracking arrays loosening without detection, and minimize the risks associated with camera tracking occlusion.

By way of nonlimiting example, in orthopedic joint reconstruction, arthritic joints are replaced with a prosthesis. Navigation may be used in a knee replacement procedure, known as a Total Knee Arthroplasty (“TKA”), for instance, in which an arthritic knee joint is replaced with a prosthesis. In a knee replacement, a series of bone resections are made to accommodate the placement of implants.

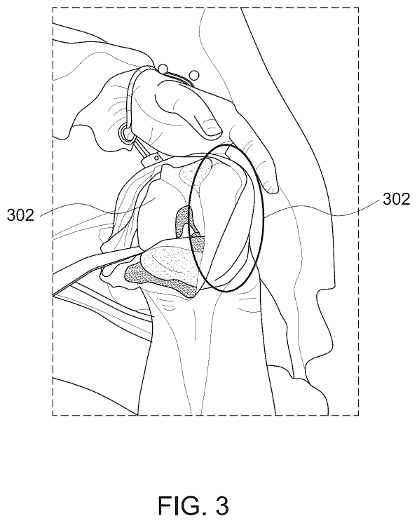

Object tracking involves determining the pose of an object and updating this pose in real-time. In the context of tracking without markers—for instance using RGBD, structured light, radar, and/or other types of cameras as described herein—there may be several challenges associated with this process. One challenge is that, in a TKA procedure in which the surgeon must cut bone, the bone is not necessarily visible in a surgical exposure because it might be obstructed by blood or other anatomy, for example soft tissue such as cartilage as one example. FIG. 3 depicts an example in which cartilage obstructs the view to bone surface underneath the cartilage. FIG. 3 shows a surgical incision that partially exposes bony anatomy 301, such as that which would present in a CT scan, for instance, but leaves at least a portion of the bony anatomy obstructed by soft tissue (cartilage in this example) in region 302. This challenge is further compounded by the fact that soft tissue such as cartilage is generally not known or characterized preoperatively because the primary form of imaging used (i.e., CT scans) cannot detect such soft tissue. Consequently, the desired bone or other object (or portion thereof) to track during the surgical procedure is at least partially not visible via line-of-sight, and is obstructed by an unknown object (e.g., soft tissue, such as cartilage).

Some aspects discussed herein present a methodology for approximating the anatomy based on a segmented point cloud. In examples, the segmented point cloud is produced by post-processing a point cloud that is generated using RGBD, structured-light, and/or other camera/sensor-based depth and image sensing technology. The post-processing of a point cloud so generated can classify and cluster regions by type (identifying anatomy and identifying the extremities or borders of such anatomy, for instance) to thereby segment the point cloud. A process also develops a tuned anatomy mesh template. The anatomy mesh template may be an initial 3D model of anatomical portions (which could include bone and/or soft-tissue anatomy). The 3D model may be a parametric 3D model. In any case, the 3D model can be deformed. For instance, the anatomy mesh template can then be deformed to correlate/match to the shape of a point cloud, for instance the segmented point cloud, such that the deformed anatomy mesh template (which may be referred to interchangeably herein as an “anatomy mesh” or “anatomy model”) provides a close approximation of the actual patient anatomy. In other words, the anatomy mesh template can be deformed to correlate with what is reflected by the segmented point cloud, representing the current pose of actual patient anatomy. With this anatomically-accurate anatomy model, which as noted could include portion(s) corresponding to soft tissue (e.g., cartilage) and portion(s) corresponding to bone, the pose of the actual patient anatomy can be determined using any of various methodologies. Hence, provided herein is a methodology to create an anatomy model based on a high-density point cloud (high number of surface points) that closely corresponds to the actual patient anatomy. Meanwhile, the position/pose of the patient anatomy can be tracked with the camera(s) used during a procedure to provide updated point cloud data that reflect any such changes, and the changes can then be reflected by updating the synthetic/virtual anatomy model. In some examples, the anatomy model includes only portions corresponding to bone, without portions corresponding to cartilage or other soft tissue.

To facilitate this, aspects can include:
1) isolating the point cloud(s) to the anatomy of interest to minimize computational demand (e.g., point cloud(s) of the knee is/are desired but point cloud(s)/cloud portions for other objects that are not visible to the cameras may be deemed a waste of computational capacity and could increase latency);
2) segmenting the anatomy of interest (in the case of a TKA, this might include segmenting the tibia and related tibial bone and tibial cartilage, and segmenting the femur and related bone and cartilage) in the isolated point cloud(s). Segmentation is the algorithmic process of, in this context, identifying anatomy and identifying the extremities or borders of such anatomy;
3) Deforming the anatomy mesh template (e.g., a femur and tibial bone mesh that includes the cartilage to correlate to the segmented point clouds(s) and thus form the anatomy model; and
4) tracking the real-time position of anatomy represented in the anatomy model based on continual updating of the point cloud described.

Aspects can provide markerless tracking with an imageless workflow (i.e., no preoperative imaging, for instance a pre-operative CT scan, necessary). Alternatively, aspects can provide markerless tracking when a known preoperative model, for example a CT scan, is available/present. In such situations, if an image-based workflow is available, only a mesh of the cartilage may be necessary (as the mesh of the bone portions can be readily established from the CT scan). The anatomy mesh template in this situation could be a cartilage mesh and can be deformed and composited with anatomical parameters of the involved bone(s), as reflected/provided by the preoperative image/information, to generate a bone and cartilage model (as the anatomy model) that is anatomically representative. In other words, the bone portion of the anatomy model can be generated from the CT scan, for instance, and the cartilage and/or other soft tissue portion can be generated by deforming a cartilage/soft tissue mesh based on point cloud information.

Computational demands associated with performance of aspects described herein can cause latency. A challenge arising from tracking objects without markers (“markerless tracking”) relates to the challenge of efficiently processing and managing the large amounts of data captured and necessary for high accuracy tracking. Markerless tracking requires processing large amounts of data (sometimes from multiple camera streams), which can introduce latency that renders the position outputs unusable (for example, the object has moved beyond an acceptable error threshold during the time between sampling the data and estimating the pose of the object). Two possible remediations include 1) speeding up the processing time so that the tracking outputs correspond more closely to the actual, real-time object pose and/or 2) fixating the object to minimize the amount that it can move during tracking. These two options can each individually and/or collectively improve the usefulness of pose estimations for objects using markerless tracking methods. Algorithm(s)/processing can be run/executed in parallel and have a different periodicity, which may, by way of nonlimiting example, be faster than other approaches. Position outputs from higher frequency/less computationally demanding tracking methods can be combined with position outputs from aspects described herein with smoothing functions. Tracking algorithms can leverage devices that provide depth information, optionally combined with image information. RGBD cameras are an example of such devices that combine images with depth information, though other device(s) providing image information correlated to depth information may be used. One such other example is a structured-light camera.

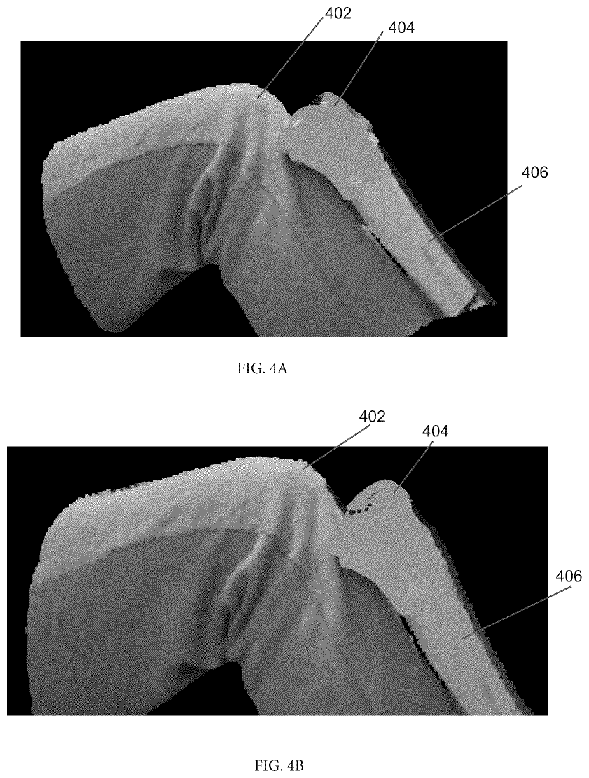

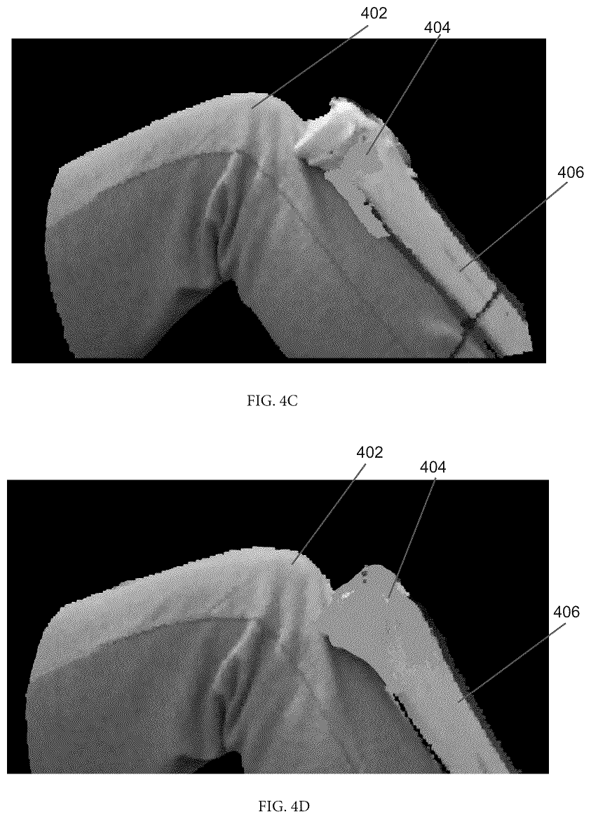

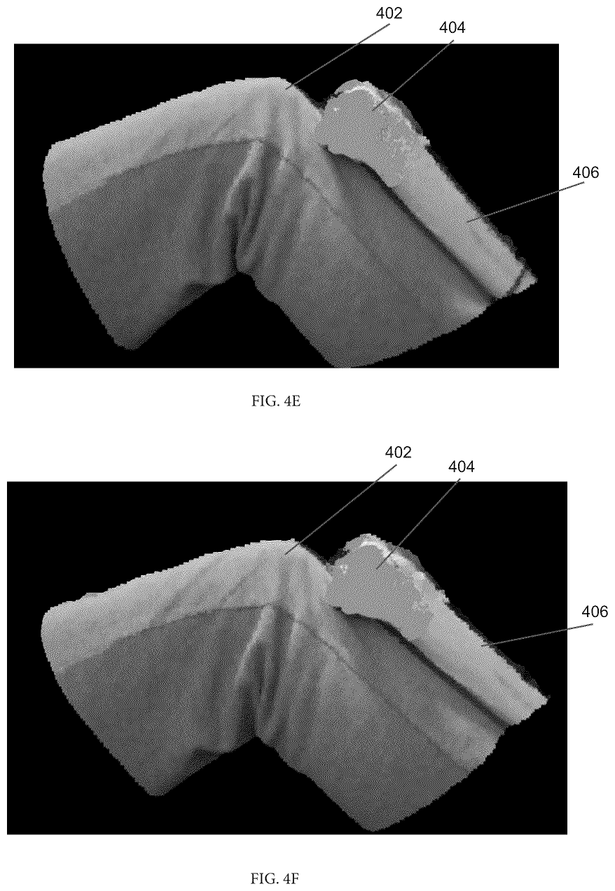

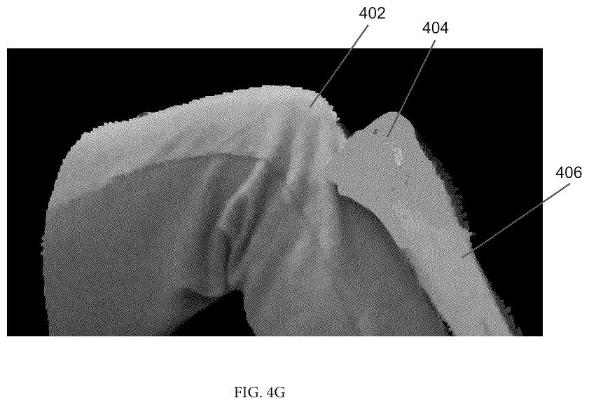

The algorithms used may include a segmented image and anatomic tracking using human pose estimation. For example, segmentation and human pose estimation algorithms (OpenPose 3D or similar) can be used for gross position tracking. FIGS. 4A-4G present an example depiction of anatomy pose tracking in accordance with aspects described herein. Specifically, FIGS. 4A-4G show the progression in pose of a knee 402, with an imposed graphical element 404 on a superior/proximal portion of the tibia 406.

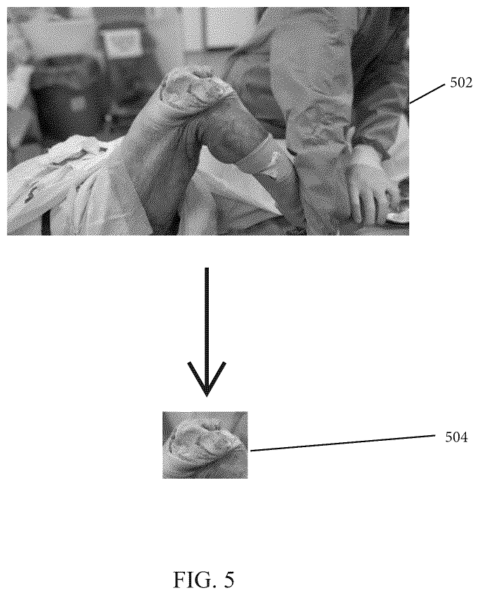

Various approaches that may be used individually and/or collectively are presented herein to help expedite processing time so that the tracking outputs correspond to the actual object pose. As an example, sensor (e.g., imaging) data can be preprocessed. An example of such preprocessing is cropping, which involves removing unwanted pixels (or other sensor data) to focus only on desired object(s)/area(s) of interest. The effect is to decrease the size/volume of data for easier and faster processing that follows. FIG. 5 depicts an example of image cropping, in which initial image/image data 502 is cropped to produce cropped image data 504 that includes the area of interest and omits areas not of interest. The file size and/or amount of data space consumed decreases in this example from about 320 kilobytes to about 59 kilobytes.

Cropping can be applied to a point cloud to reduce the number of points included in the point cloud. Generating a point cloud, especially from multiple camera streams, and working with the generated point cloud can be computationally demanding. Avoiding computational efforts to generate/process point cloud(s), or portions thereof, for objects that are not relevant for executing the procedure can be useful. Therefore, it may be desirable to cull, crop, modify, or otherwise manipulate portions of the point cloud data to avoid processing of data that is/are not relevant. This enables the processing to focus on data corresponding to the anatomy of interest, which is a subset of the entire scene.

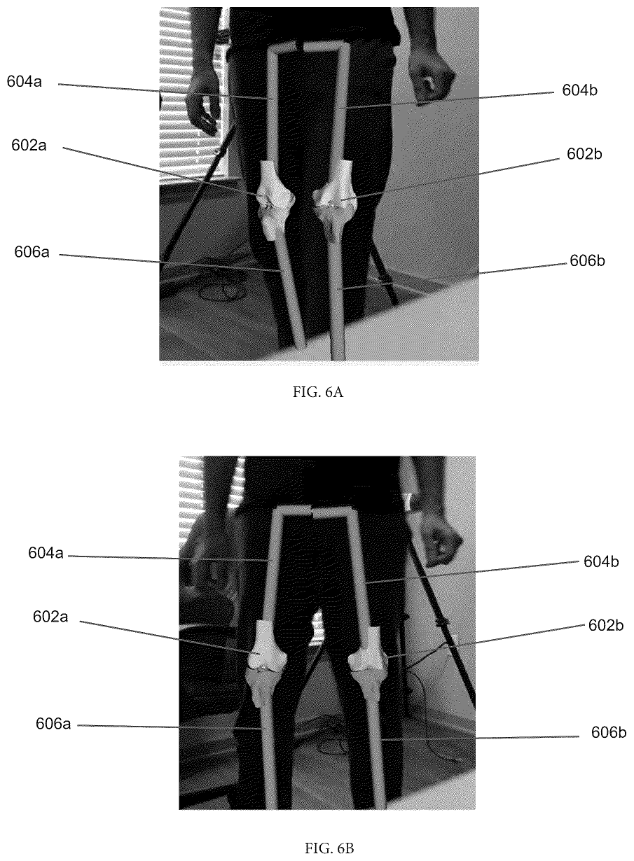

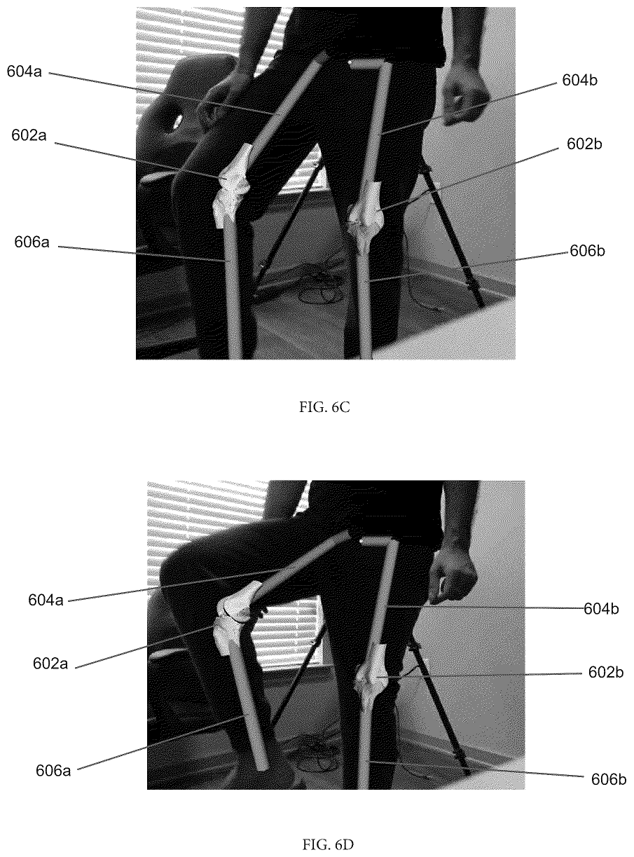

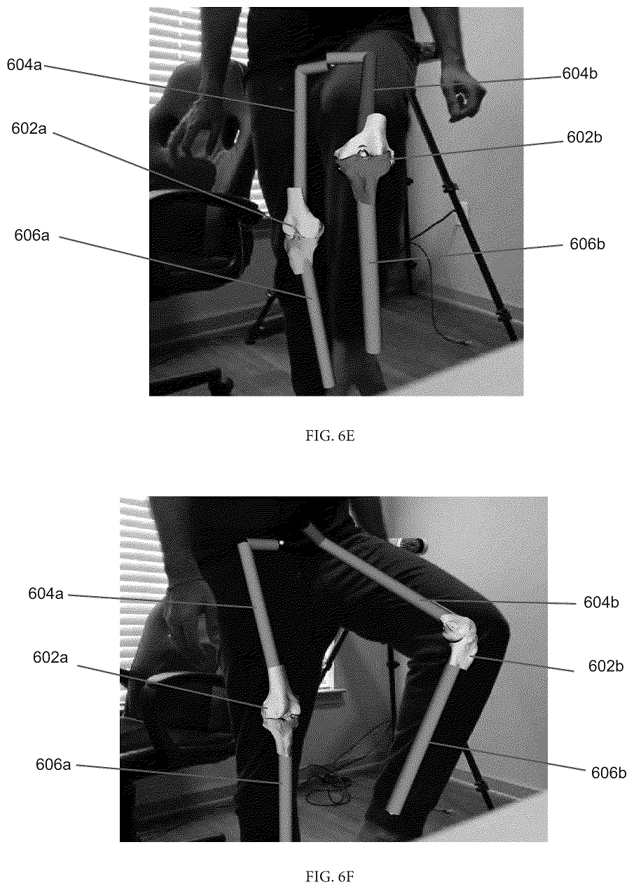

One example of this is human pose estimation using a lying pose dataset cloud to locate the knee. An example of such an algorithm is the OpenPose 3D algorithm (or similar) that relies on tensors and optimization through deep reinforcement learning. A process presented herein can use a pose estimation algorithm to identify the gross knee joint position. A thermal camera (as an example) may be used to locate the joint if the anatomy is obstructed, for example obstructed by a drape. By way of specific example, human pose estimation can be used to estimate the gross knee position and isolate this anatomy. Note that these algorithms may be able to estimate the gross position of such anatomy despite the obstruction from clothing. FIGS. 6A-6F present another example depiction of anatomy pose tracking, in accordance with aspects described herein. FIGS. 6A-6F depict the progression in pose of knee joints 602a, 602b and associated tibia and femur bones, with imposed graphical elements 604a, 606a, 604b, 606b corresponding thereto.

Another example of preprocessing is depth filtering in which camera(s), such as RGBD camera(s) and/or structured light camera(s), return the distances from the sensor to various objects in a scene and filtering is applied based on those depth distances. If the position of the camera(s) and general distance from the object is known, processor(s) could filter objects based on their depth, for instance to filter in or out objects within, outside, less than, or more than, acceptable depth value(s)/range(s).

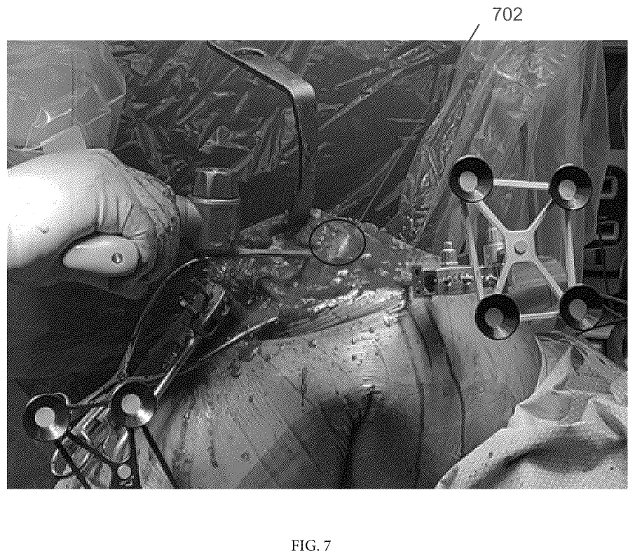

Another example of preprocessing is color filtering. Objects of interest may have color uniformity—for example, human anatomy may be distinguishable from other objects based on color. Color filtering algorithms can thusly be used to filter out objects that are not of interest. The anatomy of interest may have a distinct color, which could facilitate color-based filtering in which that anatomy is filtered in (included) or filtered out (excluded) based on detecting the anatomy by its color. FIG. 7 depicts an example in which color-based filtering is used to identify anatomy of interest by filtering an image, in accordance with aspects described herein. Referring to FIG. 7, the anatomy of interest 702 might present as lighter (whiter) and/or having a less red color than other features depicted, and this could be used to distinguish and therefore identify/crop/filter out image data of anatomy that is not of interest.

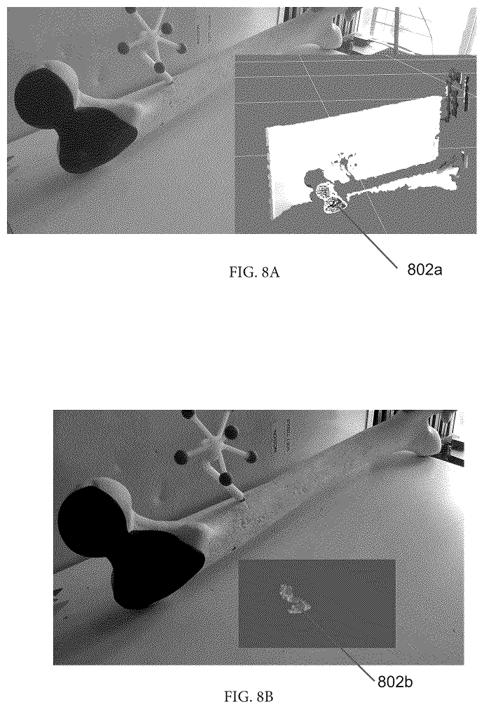

As another example, FIGS. 8A-8B depict an example in which color-based filtering is used to filter an image, in accordance with aspects described herein. Referring to FIGS. 8A-8B and comparing between the two, color filtering may be used to filter the image for anything that is, or is not, black and/or similarly could be used to filter the image for colors that are not human anatomy. FIGS. 8A and 8B show filtering to include points 802a, 802b, respectively, detected in the black area of the femoral head and neck.

Preprocessing can also implement bounding boxes. In the context of image analysis, a bounding box is often used to locate and identify objects within an image. It provides a way to define the spatial extent of an object, allowing algorithms to extract features or perform further analysis on the object within that region. The coordinates of a bounding box are typically represented as (x, y, width, height), where (x, y) denote a perimeter point, such as the top-left corner of the box, and width and height define the dimensions of the box. By utilizing bounding boxes, algorithms can localize and identify multiple objects within an image or video stream, enabling tasks such as object recognition, classification, or tracking. Methods for creating bounding boxes are well established and include object localization using deep learning models, edge detection and contour analysis, template matching, and region proposal methods, among others. In an example embodiment, a bounding box may, for example, encompass the knee. In connection with this, distance-based bounding box algorithms could be used to reduce the range of search for the object features (features of the knee) and thereby conserve computing resources.

Regarding generation of a high-density point cloud from one or more camera streams, the density of the point cloud refers to a number of points per unit of area. A relatively dense point cloud includes a relatively high number of points of an object of interest being captured. For instance, a dense point cloud could be a point cloud that significantly exceeds the number of points that would be captured in point-based registration. In an example of point-based registration, 80 points per one unit of surface or image area may be captured, while a dense point cloud could, for example, include 500 or more points per one unit of surface or image area. In some embodiments, thousands or millions of points, and in some cases more than 5 million points, are captured for accurate anatomical pose and dimension. FIG. 9 depicts an example presentation of a high-density point cloud of patient anatomy, in accordance with aspects described herein. The high-density point cloud 902 of anatomy is specifically modeling a portion of a patient's spine, in this example. The point cloud 902 is shown imposed on a graphical display over an image of the patient's spine, and more specifically is located imposed over the portion of the spine that the point cloud 902 models.

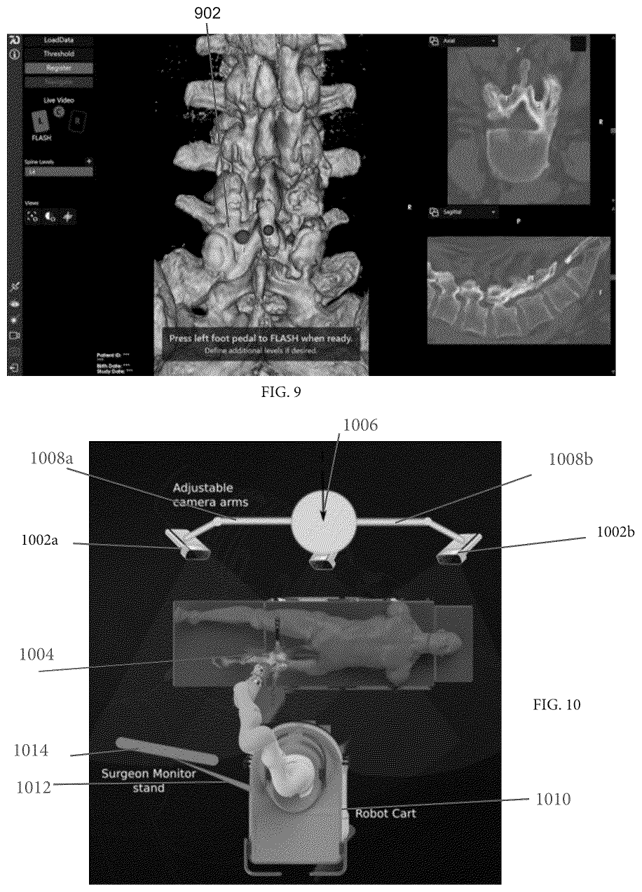

The accuracy of tracking may be dependent on the number of points captured, and the number of points captured can be a function of the number of cameras used. One or more cameras may be positioned around the operating theater. FIG. 10 illustrates an example in which a plurality of cameras (1002a, 1002b) are positioned around the operating theater, and capture multiple different views of the anatomy of interest. By way of non-limiting example, multiple cameras are positioned around the patient knee 1004, as shown in FIG. 10. Also shown is a robot cart 1010 holding components of the robotic system, including a computer system, for example, and a monitor stand 1012 securing a monitor 1014 for displaying graphical user interfaces for use by a surgeon and other users. Also shown is a camera mounting apparatus 1006 with adjustable cameral arms 1008a, 1008b for holding/mounting cameras 1002a, 1002b. Each such camera produces a respective camera stream (i.e., video; sequence of images/image data). In the case of multiple cameras, image data of the camera streams produced therefrom are combined to create a 3D representation of the object(s)/anatomy. By way of example, a bundle adjustment algorithm and/or perspective-n-point algorithm is/are used to combine the streams to create the 3D representation. In general, processes for combining the camera image streams can sync the streams and combine them to render object(s) in three dimensions.

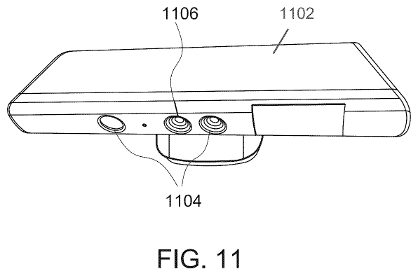

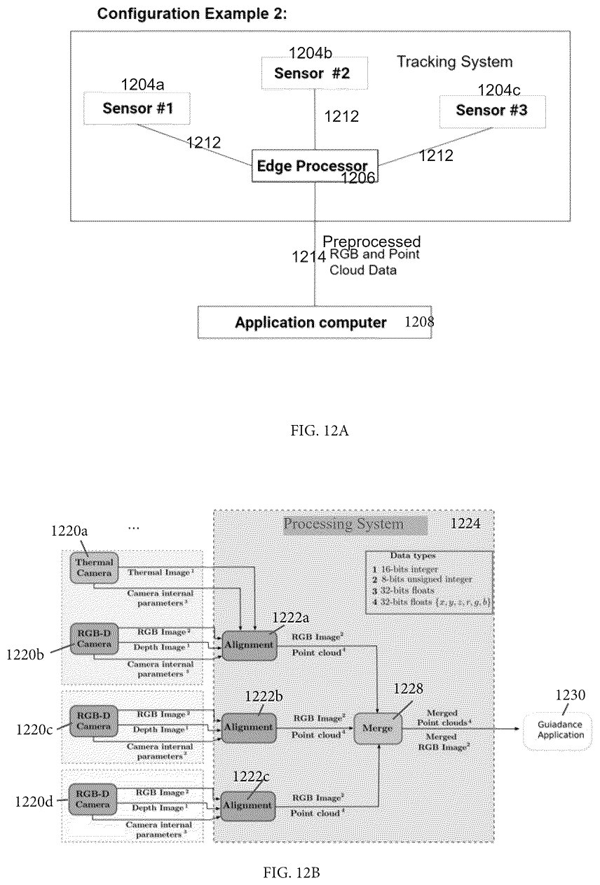

As noted, RGBD and/or structured-light scanners/cameras may be preferable to capture images and depth of features of those images. RGBD cameras can incorporate an RGB camera together with depth sensor(s). FIG. 11 depicts an example camera with depth sensing capability. Specifically, RGBD camera 1102 depicted in FIG. 11 includes 3D depth sensors 1104 and an RGB camera 1106.

Segmentation is another example of preprocessing that can be performed. Segmentation is, as noted above, a computational process that can define/identify different objects of interest or regions in an image. Generally it describes the process of dividing an image into different regions based on the characteristics of pixels to identify objects or boundaries to simplify an image and facilitate more efficient analysis thereof—for example, partitioning/segmenting cartilage and bone, partitioning/segmenting the femur from the tibia, etc. Various segmentation methods exist, including thresholding, edge-based segmentation, region-based segmentation, and contour-based segmentation, any combination of these methods, and/or machine-learning methods.

When dealing with an anatomical point cloud, the generated anatomical point cloud can be segmented to identify objects and object boundaries. In the context of a TKA, it may be desired to segment the primary anatomy of interest, i.e., femur, femur bone, femur cartilage, tibia, tibial bone, and tibial cartilage. By way of example, a machine learning-based, self-supervised segmentation approach may be used (advantageously requiring less data) and/or an encoder-decoder with generative constraints may be used to accomplish the segmentation.

Preprocessing could also perform registration, referring to the process of returning the pose and position of the object(s) of interest. Generally, registration in this context seeks to correlate a pre-operative dataset to an intraoperative dataset in order to infer the real-time position of the anatomy reflected in the preoperative dataset. This is sometimes described as registering the position of the bone (or other anatomy).

The above approaches for filtering/preprocessing are just some examples; others are possible. For instance, machine learning could be used to find the desired object(s) in the scene and crop around them. Additionally or alternatively, a small object could be introduced into the anatomy to identify the portion(s) of interest in the scene. These additions/alternatives are provided by way of non-limiting example.

In embodiments, preprocessing is accomplished by way of edge processing of image data. Edge processing, also known as edge computing, refers to performing data processing and analysis ‘closer’ to the sensors or other inputs that collect/provide the image data for processing. This typically involves deploying computing resources in proximity to the data sources. Frequently these computing resources are optimized to execute dedicated tasks with high efficiency, and may run lighter operating systems and/or may be optimized to perform focused tasks extremely efficiently. Proximity to the data source enables faster overall data processing, reduced latency, and enhanced efficiency by minimizing the need to transmit large amounts of data to a central processor that may have a higher computing burden because it is performing multiple functions. Edge processing is generally a more decentralized approach to processing data closer to the data source for improved efficiency and real-time processing capabilities.

In embodiments, sensors may include one or more cameras, for instance infrared, RGBD, thermal, and/or structured light cameras. Instead of transferring all of the data to a central computing system that is running other applications, such as a system running the surgical/robotic guidance or navigation application, aspects can perform preprocessing of this collected data with computing resources (“edge processors”) dedicated to the tasks of preprocessing, for instance dedicated to performing one or a plurality of the preprocessing approaches discussed above, and sending the preprocessed image data to downstream system. In this manner, edge processors could perform multiple functions at or nearer the data source to reduce the computations required by a central processing computer, and/or reduce the amount of data needed to be processed. A used herein, an edge processor refers to computing device(s)/system(s) that sit between the data source(s) and other systems that are running the guidance application, navigation application, or other applications to process the preprocessed image data.

FIGS. 12A-12B depict example environments employing edge processing to facilitate anatomical tracking, in accordance with aspects described herein. Referring to FIG. 12A, three sensors (1204a, 1204b, 1204c) of a tracking system 1202 provide data via wired/wireless data communications paths 1212 to an edge processor 1206 of the tracking system 1202. The edge processor 1206 receives the sensor data, which may be RGB and point cloud data for instance, and performs preprocessing of the received sensor data. The data resulting from the preprocessing (i.e., ‘processed data’) is then provided via a wired/wireless (e.g. Wi-Fi, 5G, etc.) data communication path 1214 to an application computer 1208 for further processing. In this example, the data transferred from the edge processor 1206 to an application of the application computer 1208 may contain, by way of non-limiting example, a scene's stitched segmented RGB data and segmented point cloud.

FIG. 12B depicts another example environment employing edge processing to facilitate anatomical tracking. Here, four sensors/sensor devices-a thermal camera 1220a and three RGB-D cameras 1220b, 1220c, 1220d—provide data to three alignment processors 1222a, 1222b, 1222c of a processing system 1224. Specifically, the thermal camera 1220a provides thermal images and camera internal parameters as data to a first alignment processor 1222a, a first RGB-D camera 1220b provides RGB images, depth images/information, and camera internal parameters as data also to the first alignment processor 1222a, the second RGB-D camera 1220c provides RGB images, depth images/information, and camera internal parameters as data to the second alignment processor 1222b, and the third RGB-D camera 1220d provides RGB images, depth images/information, and camera internal parameters as data to the third alignment processor 1222c. While thermal and RGB-D cameras are used in this example, the same principles could apply to structured light cameras and other suitable imaging devices.

The three alignment processors 1222a, 1222b, 1222c are hardware and/or software components of Processing System 1224 that performs preprocessing of the received sensor data, for instance one or multiple different preprocessing tasks, examples of which are discussed above and herein. In this example, each alignment processor processes the sensor data that it receives to generate RGB image(s) and point cloud(s). Each of the alignment processors can perform respective preprocessing task(s) as part of that, and as desired. For instance, an alignment processor might perform desired cropping, bounding, and segmenting, while another alignment processor might perform color filtering and segmenting. Various scenarios are possible in this regard.

As noted, the alignment processors each produce a respective set of RGB image(s) and point cloud(s). They then provide these to a merge component 1228. The merge component 1228 is hardware and/or software configured to merge data received from the alignment processors 1222a, 1222b, 1222c. In this example, the merge component 1228 (i) merges the RGB images received from the alignment processors to produce merged RGB image(s), and (ii) merges the point clouds received from the alignment processors to produce merged point cloud(s). This merging can be considered further preprocessing, and could, itself, perform any of various preprocessing discussed above, for instance cropping, bounding, segmenting, etc. Though shown as separate and discrete components of the Processing System 1224, the alignment processors 1222a, 1222b, 1222c and the merge component 1228 can be implemented by the same or different hardware/software. In an example, a single computer system executing one or more software modules implements the Processing System component. In another example, separate computer systems/devices are provided for each of the alignment processors and merge component. In yet other examples, a computer system/device implements one subset of the alignment processors and merge component, and one or more other computer systems/devices implement another subset of the alignment processors and merge component. Various implementations are possible.

Output of the Processing System component 1224 includes output from the merge component 1228, for instance merged RGB image(s) and merged point cloud(s). This output ‘processed data’, is sent to an application 1230 for use. In this example, the application 1230 is a guidance application for controlling guided robotic surgery. By way of specific example, the output of the preprocessing by the Processing System 1224 is used by the guidance application to perform further processing using the merged point cloud(s)/RGB image(s), for example processing to perform segmentation, anatomy mesh template deformation and updating, for instance based on streamed/updated point cloud data as the patient anatomy is imaged during a surgical procedure, tracking, and/or other tasks. In this manner, a guidance application is to perform a collection of tasks in connection with a guided robotic surgery that is in-progress and/or to-be-performed, the collection of tasks of the guided application is premised on input image data that the guided application receives, and this input image data is preprocessed data that has been preprocessed by preprocessing component(s), such as those of the Processing System component 1224, which itself received raw sensor data from sensor(s) 1220a, 1220b, 1220c, 1220d. Thus, one or more secondary computing systems (edge devices; 1224 in the example of FIG. 12B) that is/are not dedicated to running the guidance (or other) applications (such as navigation) may be used to preprocess sensor data. In a specific example, each alignment processor segments and registers the individual camera stream data that it receives, provides that as output to the merge component, and then the merge component merges the received data from the individual alignment processors before sending it to the guidance (or other) application for further processing.

When using structure light camera(s), there are some challenges that could arise. First, the camera(s) tend to have relatively high latency because they project images. Some may be able to capture only as few as two images per second. Second, though tracking with multiple structured light cameras may be important for mitigating occlusion and improving accuracy, simultaneous projection from multiple structured light cameras may cause interference of the pattern projection that renders simultaneous tracking with multiple cameras a challenge. Third, the lighting in the area (e.g., operating room) may interfere with the projected image, especially if it has a similar wavelength to the light projected by the camera(s).

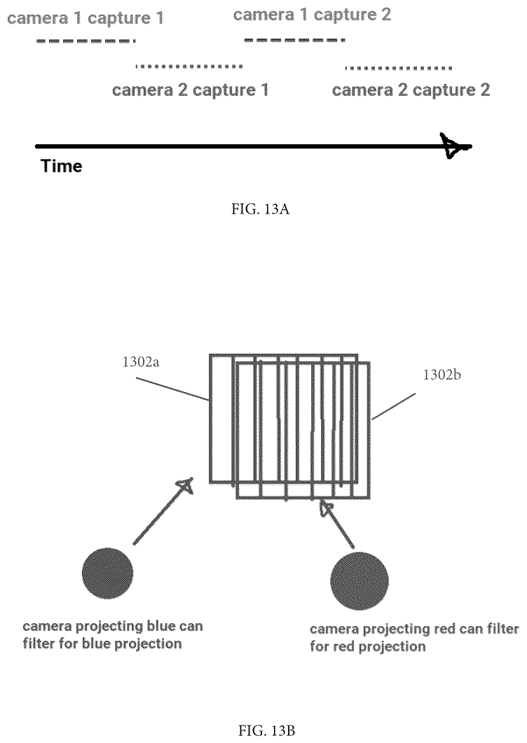

FIG. 13A-13B depict example differentiated structured light camera projection approaches, in accordance with aspects described herein. To help improve latency, and in accordance with aspects herein, tracking with multiple cameras includes staggering the image capture periodicity as shown in FIG. 13A. In other words, the image capture of each of the multiple cameras can be staggered such that the projection from each camera does not interfere with the projection from any of the other camera(s). By way of a non-limiting example, and assuming two cameras, the periodic capture from the first camera is staggered with the periodic capture from the second camera so that neither captures when the other captures. FIG. 13A depicts this situation, in which the sequential captures (1, 2, etc.) by camera 1 over time are spaced apart and separated by the sequential captures (1, 2, etc.) by camera 2 so that when one camera captures, the other does not. This can help address issues related to system latency pertaining to structured light cameras, as one camera can capture an image while another one (or more) processes image data before next image capture. This could allow for more images captured per unit of time.

As noted, simultaneous image capture (by multiple cameras) of the projected images can be problematic. Aspects discussed herein therefore propose, in a second approach, as shown in FIG. 13B, using a different color for the projected images from each respective camera being used. For example, one camera may project an image in first color (such as blue), and another camera may project an image in a second color (such as red) that is different from the first color. An RGB camera may be used as the sensor in each camera, so filtering by color may render it possible to mitigate interference of the projected images from multiple cameras. Referring to FIG. 13B, one camera can project blue light images 1302a and filter for blue projection, while another camera can project red light images 1302b and filter for red projection. This may be possible with three or more cameras, in which each camera projects and filters for a respective color different from the colors used by the other cameras.

As for ambient lighting in the area (e.g., operating room) and interference with the projected image from a structured light camera, the wavelength of the projected image can be altered to overcome this. For example, a blue, red, etc. projection can be used to maintain visibility of the projected image.

As described above, one challenge of tracking may be latency. While optimized data processing as described herein can help, reducing the amount of movement that an object of interest can move at critical times may offer further benefit. Structured light sensors, for instance, may have high accuracy but may also have high latency.

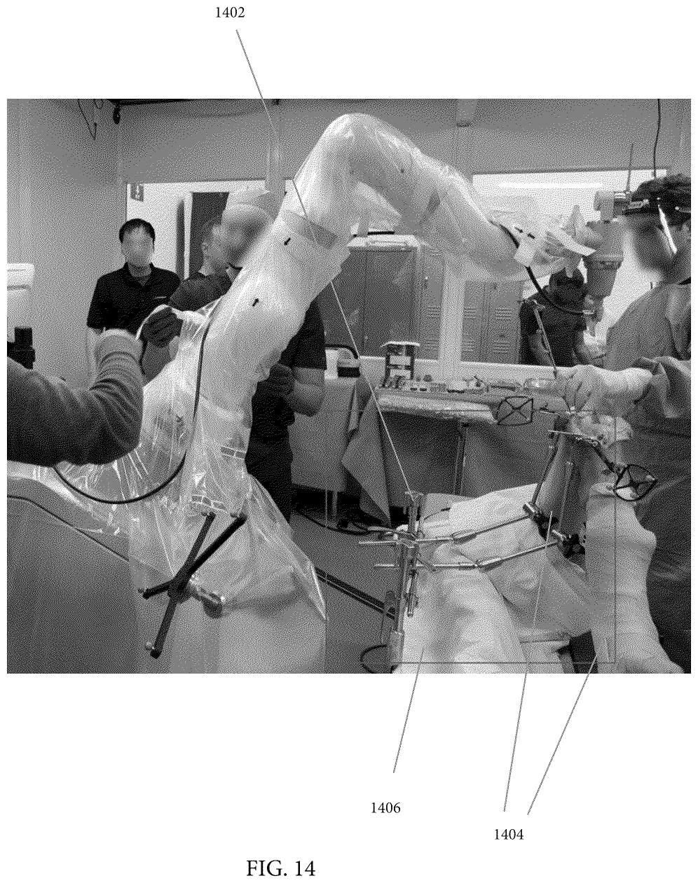

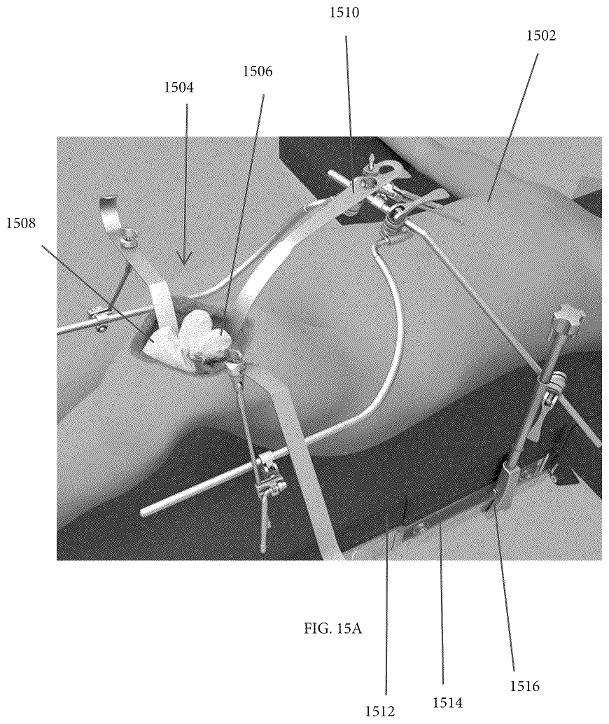

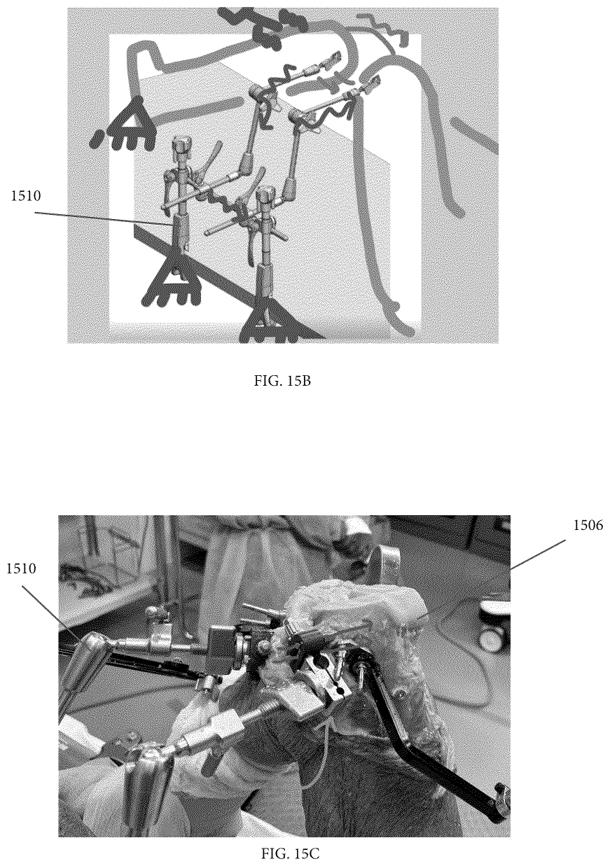

One approach described herein to help remediate latency inherent to markerless tracking is fixating tracked object(s) (in the sense of rigidly securing/fastening the object(s)) so that movement thereof is minimized between point samples. An example of a specific embodiment of fixation of objects of interest for markerless tracking, and in specific embodiments for TKA applications, is shown by FIG. 14. Various anatomical feature(s) that are object(s) of interest, such as bones 1404 (e.g. a femur and/or tibia), can be rigidly fixated (“fixated” is used interchangeably with the word “fixed” herein) to a stationary object like the surgical table or robot 1406, as examples, by way of a fixator apparatus 1402. By way of non-limiting example, when fixated, the bone(s) 1404 could be constrained to move less than some distance, for instance 1 mm, during cutting or other surgical procedures. Such constraints could render the latency of markerless tracking less problematic. In addition, if movement above a threshold (such as 1 mm) is observed based on the tracking during a surgical action (maneuver, step, cut, or the like), then in some embodiments the robot system can be commanded to halt/stop/pause the action, at least temporarily. For instance, if during performance of a cut by a robotic cutting tool it is observed that the anatomy moves 1 mm or more, the control system of the robotic cutting tool can cause the cut to be at least temporarily aborted pending user/doctor intervention.

External fixation in surgery is a technique used to stabilize bones or other objects by placing fixation devices externally on the body. These devices typically consist of pins, wires, and/or screws that are inserted into the bone and are then connected to an external frame, often made of metal rods, that holds the bone fragments in the desired position. An external fixator such as 1402 rigidly attaches the anatomy to devices/objects that remain fixed in position, such as the surgical table or robot cart, as examples.

Provided herein to facilitate markerless tracking, for instance in scenarios in which camera(s) may have relatively high latency, is an approach to rigidly fixate the object(s) (e.g., bone(s)) of interest to something that remains stationary. In an example specific morphology, bones are fixated using bone screws and devices that rigidly stabilize the bones to the surgical table, surgical cart, or other objects that are not expected to move during a surgical procedure. Accordingly, in some aspects, rigid fixation of object(s) of interest to a stationary object such as a surgical cart, table, or robotic surgical device is provided. This constrains motion/movement of the object(s) in order to avoid or remediate issues related to latency. By this is meant that a level of assurance is provided that anatomy has not moved significantly between the time a camera images the anatomy and determines the position of the anatomy in space and a time a robot performs an action based on that position of the anatomy. In specific embodiments, such rigid fixation is provided in an environment in which markerless tracking as described herein is also provided, for instance in connection with a TKA. These aspects differ from other approaches that attempt real-time tracking but do not constrain movement of the object(s) of interest.

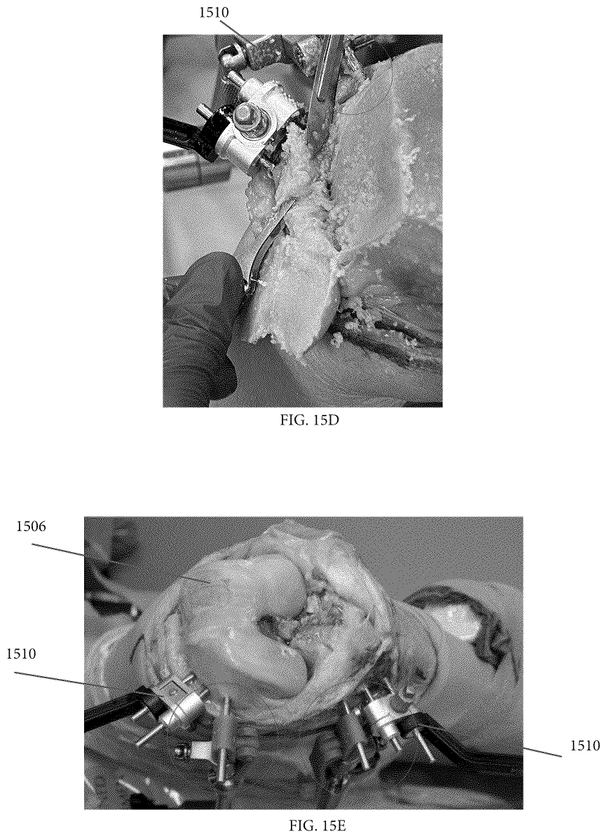

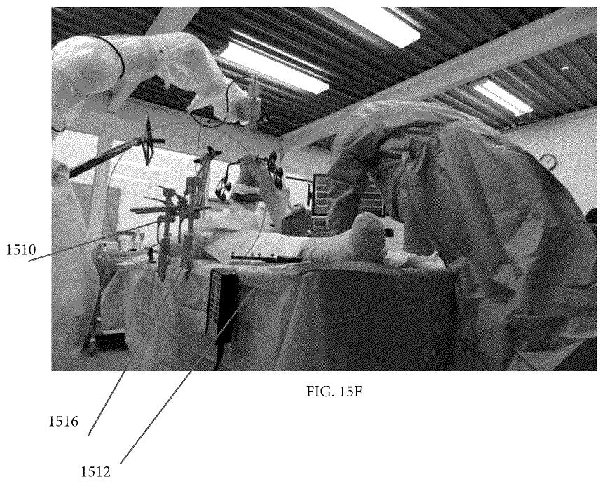

FIGS. 15A-15F illustrate additional examples of fixing objects to facilitate markerless tracking in accordance with aspects described herein. Referring to FIG. 15A, patient 1502 is undergoing a surgical procedure, specifically a TKA on the patient's knee joint 1504, which involves (at least) the patient's femur 1506 and tibia 1508 as anatomical objects of interest. The femur and tibia are shown partially exposed and at least a portion of which are to be tracked, for instance via markerless tracking as described herein. To facilitate this, fixation device 1510 is provided to rigidly fixate this anatomy of interest relative to other object(s), in this example to the surgical table 1512, specifically to a metal frame 1514 thereof. In this example, the fixation device 1510 includes clamps 1516 (one of which is shown in FIG. 15A) that clamp to the frame 1514 at (at least) three positions in this example.

Additional examples are shown in FIGS. 15B-15F using similar reference numerals to those of FIG. 15A to denote similar objects/components.

In examples, external fixation devices for fixating target anatomy of interest for targeted surgical procedures may be provided together with systems/components for markerless tracking as described herein.

Another aspect of the disclosure relates to soft tissue balancing, for instance a soft tissue balancing performed during a TKA prior to cutting. Soft tissue balancing in this context refers to the process of achieving proper tension and alignment of the soft tissues around the knee joint. A goal is to create a stable and well-aligned knee after the damaged or diseased joint surfaces have been removed and replaced with an artificial knee implant. Soft tissue balancing can be crucial in total knee replacement surgery to help ensure proper alignment, stability, and function of the knee joint. It involves assessing and adjusting the tension in the ligaments and muscles surrounding the knee to achieve a balanced joint. If the soft tissues are not appropriately balanced, it can lead to issues such as joint instability, poor range of motion, unequal weight distribution, and increased wear and tear on the implant.

During the procedure, the surgeon carefully assesses the ligaments and soft tissues to determine the appropriate tension. This can be done through a variety of techniques, including manual manipulation, use of tensioning devices, and intraoperative measurements. The surgeon may release tight tissues or tighten loose tissues to achieve the desired balance. Generally, this consists of manipulations through a range of motion, from flexion to extension. During the manipulations, measurements of the relative distances between the femur and tibia are used to determine the optimal balance. With conventional methods, these distances are calculated based on optical tracking of the relative positions of rigid arrays fixated into the femur and tibia respectively.

In the current state of the art, the gaps that are used to achieve tissue balance are measured optically with cameras that track rigidly affixed arrays. The positions of these arrays are registered to the anatomy. One benefit of tracking arrays is that they are rarely obscured by anatomy, especially when the knee is in extension.

However, to achieve markerless tracking, it is necessary to have sufficient visibility of the exposed knee anatomy. When the knee is in flexion, a surgeon can sublux the patella so that both femoral condyles and significant portions of the tibia may be visible. However when the knee is in extension, the patella cannot be easily subluxed and the knee anatomy may be obscured. Limited visibility of the anatomy can make markerless tracking difficult as discussed previously. Markerless tracking may require a method of tracking the bones when anatomy is obscured. These methods could also be applied for tracking during cutting, but a significant use case is during registration (the pins are unlikely to move during gap balancing but may move under stresses during cutting).

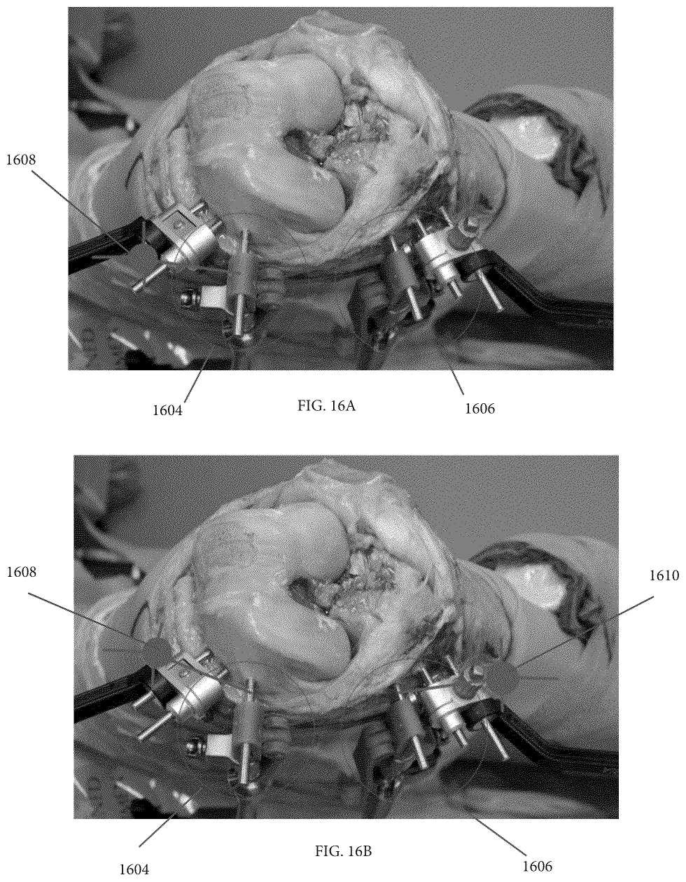

Proposed herein is the use of external fixation pins (previously described) doubling as tracking arrays if needed (especially during soft tissue balancing). The pins themselves could have features that allow for a camera to determine their pose, and/or the pins could have attachments that allow for a camera to determine their pose. Referring to FIGS. 16A-6B, which depict example external fixation pins also serving as tracking arrays in accordance with aspects described herein, 1604 and 1606 encircle pin structures of the fixation devices. The pin structures themselves could incorporate features that may be tracked. Additionally or alternatively, attachments 1608, 1610 to the pin structures could be provided for tracking. The position of these pins (and/or attachments) relative to the anatomy could be determined with the knee in flexion. Once the pins (and/or attachments) are registered to the anatomy, the determination of their orientation can inform tracking the position of the femur and tibia accordingly.

ANATOMY MESH DEFORMATION: For an imageless approach in which a preoperative patient-specific model, for example a CT scan, is not available/present, a predefined anatomy mesh template/model may be used and then deformed in accordance with aspects discussed herein. The anatomy mesh template can have vertices and faces. In the case of a TKA, the anatomy mesh template could potentially have/provide region(s) for bone and region(s) for soft tissue (which for purposes of simplicity and this example will be referred to as just “cartilage”)—for instance, regions for each of the tibia and femur and regions for the cartilage associated therewith. There are thus four objects of interest in this example: With respect to the tibia, one object is tibia bone and another is tibia cartilage. With respect to the femur, one object is femur bone and another other is femur cartilage. In general, an anatomy mesh template/model template can include/incorporate different region(s) corresponding to different type(s) of anatomy (e.g., cartilage, bone) and in the case of a TKA, the template can provide two regions (cartilage and bone) corresponding to each of the tibia and femur anatomical elements. If instead a preoperative patient-specific model, such as one of bone anatomy produced based on an available CT scan, is available, then this can be combined with a predefined anatomy mesh template of other anatomy, for instance of cartilage. Thus, if a preoperative model (such as one provided by a CT scan) is available, then that information can be used to create a 3D model of some portion(s) (e.g., the bony anatomy portion) of the anatomy model for the patient, and it is not necessary and/or desired to deform those portion(s). On the other hand, because some preoperative models (such as provided by a CT scan) do not ‘see’ cartilage, various potentially important properties of the cartilage, such as thickness, may be unknown, although other properties, such as cartilage boundaries, may be identified. In these situations, an anatomy mesh template of the cartilage can be provided for deforming (consistent with what is reflected by the segmented point cloud, for example) in order to conform within the constraints informed by the (known) boundaries and properties of known anatomy (such as bone) as informed from the pre-operative imaging. In these situations, the known anatomy could be reflected as generally non-deformable parts of the anatomy mesh template which also includes deformable cartilage portions, or could be provided directly as-is in the final anatomy model after being combined with a deformed cartilage mesh template for the patient. The deforming of the anatomy mesh template can focus on deforming/fitting the portion of anatomy that is unknown to the portion of the anatomy that is known, for instance in the case of a CT scan, fitting the portion of the anatomy mesh template corresponding to cartilage to the (accurate) model of the bone informed by the CT scan.

A goal may be to deform the anatomy mesh template through an optimization loop to provide some optimization, such as a minimization of a distance, and in some examples a chamfer distance, between the mesh template and the segmented point cloud discussed above. In the context of a chamfer distance optimization or other distance optimization(s) between the anatomy mesh template and segmented point cloud, this may be performed using an algorithm, for instance the Stochastic Gradient Descent algorithm, taking into consideration optimization loss gains for each anatomic material (e.g., cartilage and bone). This will converge eventually to the closest deformation of the anatomy mesh template into the segmented point cloud, fitting the point cloud and finding the transformation between the relevant anatomy and the data provided by RGBD camera, structured-light camera, or other depth-and-imaging device(s) being used. The result is an accurate anatomy model.

Segmentation may be extremely important for deformation of the anatomy mesh template. For example, it is desired not to deform a bone portion that is unobstructed by cartilage (i.e., that can be seen directly) since the surface point cloud for that portion should be taken as an accurate reflection of the unobstructed anatomy. Instead, it may be desired to deform portions (e.g., bone regions) that are obstructed by the cartilage (or other obstruction(s)) since the point cloud is not taken as being an accurate reflection of that anatomy. In an example deformation, deformation ‘weights’ can be introduced and used, in which different regions have varying sensitivity to deformation depending on how they are identified by the segmentation. It may be desirable not to deform a bone surface that is known (e.g., based on direct line-of-sight available to the camera) to be actual bone surface. Surfaces that have a higher likelihood of being soft tissue may be more readily deformed in optimizing the mesh template to reflect true patient anatomy.

It may be possible to generate an anatomy model using a trained machine-learning algorithm whereby an anatomically representative anatomy model is generated based on the point cloud. For instance, a machine learning (ML) model could be trained to predict n-number of parameters (Encoder-Decoder architecture) to deform an anatomy mesh template more efficiently (e.g., faster and/or more accurately) than using a traditional optimization algorithm. In such examples, model input can be a segmented point cloud and model output can be a vector of values that are used to deform the anatomy mesh template to fit the segmented point cloud.

Because processing associated with the above could be computationally demanding, a simultaneous intermediate accuracy fitting could be run, with the RGBD, structured-light, or other cameras/scanners using a OpenPose 3D (or similar) algorithm with the segmented point cloud data. The pose of the anatomy can be updated with a fine fitting model and intermediate fitting model(s) that may have differing periodicity. Other approaches, potentially faster to calculate, could be used to determine a gross estimation of the position of the anatomy, with two (or more) involved algorithms being simultaneously run.

As described previously, one challenge faced is registration of the bone position (e.g., determination of the initial pose of the bone) or registration of the position of an object with respect to bony anatomy, for instance in situations where the bone is at least partially obscured by other objects, for instance soft tissue such as cartilage. FIG. 3A shows a surgical incision that partially exposes a bone 301 but leaves at least a portion of the bone obstructed by cartilage in region 302. A common state-of-the-art practice is to place rigid bone-mounted arrays that are registered to the anatomy via a point sampling approach where a point cloud of bone surface points is captured by manually sampling surface points with a sharp-tipped tracked probe of known dimensions.

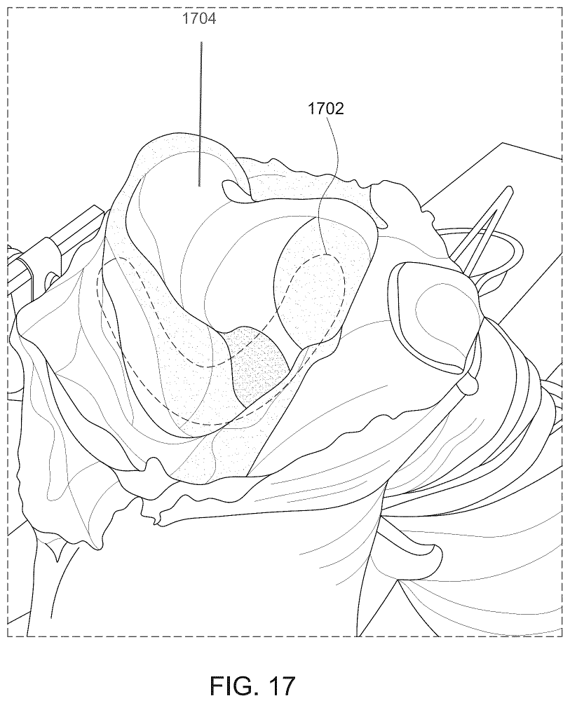

One approach to addressing the challenge of registering a bone that is partially or fully obscured by cartilage and/or other anatomy without point-based sampling is set forth as follows, and can be used to register with submillimeter accuracy, in examples.
Step 1: Initially, the process generates/captures a point cloud. As noted, a point cloud, typically maintained by a computer system in/as a data file, includes a large number of points on the surface of an object—i.e., a set of vertices in a 3D coordinate system with X, Y, and Z coordinates to define these vertices). The point cloud therefore represents the shape of the object. There are multiple acceptable ways to generate such a point cloud, for instance using structured light cameras or RGB-d cameras, as examples. In accordance with aspects described herein, an example camera used is a camera that includes laser-based ranging technology, for instance LIDAR (“light detection and ranging”; also referred to as “laser imaging, detection, and ranging”) technology that uses pulsed lasers for ranging. Such LIDAR-based camera technology can be leveraged to generate the point cloud of patient anatomy, including bone surface and soft tissue, as examples.
Step 2: The generated point cloud can then be run through a segmentation algorithm that identifies/distinguishes between visible bone surfaces and visible cartilage (or other soft tissue) surfaces, as well as anatomical landmarks (for example the most distal points of the articulating cartilage surface). An example such segmentation algorithm could be one that uses a trained machine-learning model to segment the anatomy as discussed above. The segmentation algorithm could include manual inputs, in some examples. For instance, a user could highlight cartilage and/or bone regions on a 3D reconstruction (from the point cloud) to help the algorithm with segmentation. FIG. 17 depicts an example exposed knee joint and anatomical features thereof with a highlighted anatomical region in accordance with aspects described herein. In FIG. 17, highlighting 1702, which could have been indicated, highlighted, drawn, provided, or the like, by a user, denotes a bone region of the femur 1704.
Step 3: With the segmented point cloud/of the scene that identifies bone regions and cartilage regions (as examples), the process then runs a gross registration of a reference bone model (such as a reference model of the actual bone from a CT or anatomical dataset) to the visible bone regions as reflected/detected by the segmented point cloud. The reference bone position can be, for instance, CT-based, i.e., from a reconstructed patient-specific CT scan, imageless, i.e., a generalized mesh model of the anatomy, etc., referring to the relation between different coordinates frames that exists across the point cloud, anatomy mesh template, and cameras, for instance. In any case, this provides an estimation of the pose of the reference bone model, which is an estimation of the pose of the actual patient's bone. The process can then virtually project an approximation (e.g., in the form of a mesh structure for instance) of the respective cartilage bodies (femur and tibia) (volume and surface) onto the distal femur surface and the proximal tibial bone surface, respectively, making a best effort to avoid estimated bone regions from the gross registration. The articulation of the femur cartilage grossly matches the articulating distal surface of the femur, and the tibia cartilage's flat surface grossly matches the proximal tibia surface's flat surface, for instance. The process can make generalized assumptions about the depth of the cartilage surface. For example, the process could assume that the cartilage is generally uniformly thick with selected depth(s), for instance selected depth(s) between 2 and 3 mm, inclusive. In examples, the process could assume the cartilage has variable but known depth(s) in different regions. The assumptions could be made based on prior known anatomical approximation of the cartilage thickness. It is noted that the process could be generalized to other anatomy, for example hips, shoulders, ankles or spine. In general, therefore, depth or depths for the cartilage body/bodies is/are assumed, and the process projects this cartilage body/bodies onto the CT model (or a generalized bone model in the imageless embodiment). The resulting model provides the anatomy mesh template of bone and soft tissue that may be deformed in accordance with the following step.
Step 4: The process can follow aspects described herein, for instance to deform the combined mesh model from step 3. The process can deform, minimally or not at all, assumed bone regions and allow for deformation of cartilage regions with constraint(s) commensurate with the assumed cartilage thickness(es) in the corresponding regions. The amount of respective allowed deformation can be provided as weights, and thus the allowed deformation of an assumed cartilage region may have a higher weight than the assumed deformation of an assumed bone region, for example. The end result may be an estimation as to the shape, position, and other characteristics of the full patient anatomy, including bone and cartilage. In addition, the process can use a registration method, for example a multi-hypothesis registration method, to register the full patient anatomy model to the visible point cloud from step 1 above. This provides a model of the full patient anatomy, which, once obtained, can be used with various available methods for registration to the visible scene.

Aspects described herein can be helpful for any navigated surgical procedure, and other industrial applications.

One or more embodiments described herein may be incorporated in, performed by, and/or used by one or more computer systems, such as one or more systems that are, or are in communication with, a camera system, tracking system, and/or orthopedic surgical robot, as examples. Processes described herein may be performed singly or collectively by one or more computer systems. A computer system may also be referred to herein as a data processing device/system, computing device/system/node, or simply a computer. The computer system may be based on one or more of various system architectures and/or instruction set architectures.

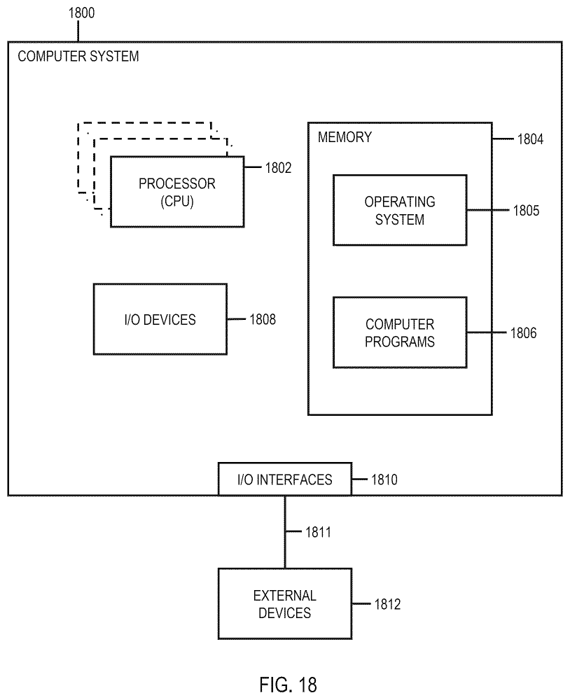

FIG. 18 shows a computer system 1800 in communication with external device(s) 1812. Computer system 1800 includes one or more processor(s) 1802, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 1802 can also include register(s) to be used by one or more of the functional components. Computer system 1800 also includes memory 1804, input/output (I/O) devices 1808, and I/O interfaces 1810, which may be coupled to processor(s) 1802 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 1804 can be or include main or system memory (e.g., Random Access Memory) used in the execution of program instructions, storage device(s) such as hard drive(s), flash media, or optical media as examples, and/or cache memory, as examples. Memory 1804 can include, for instance, a cache, such as a shared cache, which may be coupled to local caches (examples include L1 cache, L2 cache, etc.) of processor(s) 1802. Additionally, memory 1804 may be or include at least one computer program product having a set (e.g.,, at least one) of program modules, instructions, code or the like that is/are configured to carry out functions of embodiments described herein when executed by one or more processors.

Memory 1804 can store an operating system 1805 and other computer programs 1806, such as one or more computer programs/applications that execute to perform aspects described herein. Specifically, programs/applications can include computer readable program instructions that may be configured to carry out functions of embodiments of aspects described herein.

Examples of I/O devices 1808 include but are not limited to microphones, speakers, Global Positioning System (GPS) devices, RGB, IR, and/or spectral cameras, lights, accelerometers, gyroscopes, magnetometers, sensor devices configured to sense light, proximity, heart rate, body and/or ambient temperature, blood pressure, and/or skin resistance, registration probes and activity monitors. An I/O device may be incorporated into the computer system as shown, though in some embodiments an I/O device may be regarded as an external device (1812) coupled to the computer system through one or more I/O interfaces 1810.

Computer system 1800 may communicate with one or more external devices 1812 via one or more I/O interfaces 1810. Example external devices include a keyboard, a pointing device, a display, and/or any other devices that enable a user to interact with computer system 1800. Other example external devices include any device that enables computer system 1800 to communicate with one or more other computing systems or peripheral devices such as a printer. A network interface/adapter is an example I/O interface that enables computer system 1800 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 1810 and external devices 1812 can occur across wired and/or wireless communications link(s) 1811, such as Ethernet-based wired or wireless connections. Example wireless connections include cellular, Wi-Fi, Bluetooth®, proximity-based, near-field, or other types of wireless connections. More generally, communications link(s) 1811 may be any appropriate wireless and/or wired communication link(s) for communicating data.

Particular external device(s) 1812 may include one or more data storage devices, which may store one or more programs, one or more computer readable program instructions, and/or data, etc. Computer system 1800 may include and/or be coupled to and in communication with (e.g., as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 1800 may be operational with numerous other general purpose or special purpose computing system environments or configurations. Computer system 1800 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

Aspects of the present invention may be a system, a method, and/or a computer program product, any of which may be configured to perform or facilitate aspects described herein.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g., instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C#, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Although various embodiments are described above, these are only examples.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

Processes/methods claimed herein may be executed, in one or more examples, by a processor or processing circuitry of one or more computers/computer systems, such as those described herein. In examples, code or instructions implementing the process(es) are part of a module. In other examples, the code may be included in one or more modules and/or in one or more sub-modules of the one or more modules. Various options are available.

Below is a small sampling of aspects described herein.
A1. A computer-implemented method for tracking at least one object in an environment, the method comprising: (i) generating a point cloud from images/image data obtained from one or more cameras, the generating comprising: imaging, using the one or more cameras, the at least one object, wherein each camera of the one or more cameras provides a respective image stream of one or more image streams; and generating the point cloud as a three-dimensional (3D) representation of the at least one object based on the one or more image streams; (ii) segmenting the generated point cloud into a segmented object model for the at least one object, wherein the segmenting identifies the at least one object and extremities/borders of the at least one object as exhibited by the generated point cloud; and (iii) deforming a predefined mesh model, representative of at least a portion of the at least one object, to correlate to the segmented object model, the deforming providing a position/pose of the at least one object in the environment.
A2. The method of A1, further comprising: iteratively performing, across a duration of time, the generating, the segmenting, and the deforming, to provide updated positions/poses of the at least one object in the environment at corresponding points in time; and using the updated positions/poses to track position of the at one object in space over time.
A3. The method of A2, further comprising presenting a model of the at least one object on a display device and with positions/poses corresponding to the determined positions/poses of the at least one object over time.
A4. The method of any of A1 to A3, wherein the at least one object comprises anatomy of interest of a surgical patient to undergo a surgery.
A5. The method of A4, wherein the anatomy of interest comprises bone and/or soft tissue of the surgical patient.
A6. The method of A5, wherein the segmented object model is a segmented bone and/or soft tissue model for the surgical patient.
A7. The method of A6, wherein the deforming provides a deformation of the mesh model to converge to the segmented bone and/or soft tissue model to within a threshold, providing a transformation between the bone and/or soft tissue and image data captured by the one or more cameras, therefore providing an anatomically accurate bone and/or soft tissue model that includes bone and/or soft tissue, and provides the position/pose of the bone and/or soft tissue.
A8. The method of any of A4 to A7, wherein the surgery is a knee arthroplasty.
A9. The method of any of A4 to A8, wherein the tracking is without/absent preoperative patient-specific imaging/image(s)/model.
A10. The method of any of A4 to A, wherein, based on preoperative patient-specific imaging/image(s)/model being available, the mesh model is composed, for a portion of the anatomy of interest, with information from the patient-specific imaging/image(s)/model.
A11. The method of any of A1 to A10, wherein the mesh model is predefined at least partially based on based on anatomy of a population of individuals.
A12. The method of any of A1 to A11, wherein the tracking is performed without/absent relying on known marker(s) (such as fiducials or other markers) placed into the environment or adjacent/on the at least one object, for instance for optical, beacon-based, RADAR-based, or other forms of tracking of the marker(s) in the environment to track the at least one object.
A13. The method of any of A1 to A2, wherein the generating comprises aggregating image data of the one or more image streams to provide the point cloud.
A14. The method of A13, wherein the aggregating uses a bundle adjustment algorithm and/or perspective-n-point algorithm is/are used to aggregate the image data.
A15. The method of any of A1 to A14, wherein the one or more cameras comprise: (i) RGB (Red, Green, Blue); and/or (ii) RGBD (Red, Green, Blue Depth) and/or structured-light camera(s) that produce the images/image data with depth information.
A16. The method of any of A1 to A15, wherein the generating further comprises filtering out image data points corresponding to object(s)/object portion(s), such as anatomy of interest, not visible to the one or more cameras.
A17. The method of A16, wherein the filtering uses one or more of: (i) a pose estimation algorithm that estimates position of the at least one object despite line-of-sight obstruction between the one or more cameras and at least a portion of the at least one object; (ii) distance-based bounding that constrains a range of search for the at least one object in the image data; and (iii) a color filtering algorithms to filter out image data portions corresponding to one or more objects, or portions of the one or more objects, that are not of interest.
A18. The method of A17, wherein the pose estimation algorithm is an OpenPose algorithm.
A19. The method of any of A1 to A18, wherein the segmenting uses (i) a machine learning model and/or (ii) an encoder-decoder with generative constraints.
A20. The method of any of A1 to A19, wherein the mesh model comprises vertices and faces.
A21. The method of any of A1 to A20, wherein the deforming is performed with a goal to deform the mesh model through an optimization loop to minimize a selected distance between the mesh model and the segmented object model.
A22. The method of A21, wherein the optimization loop uses a Stochastic Gradient Descent algorithm.
A23. The method of any of A1 to A22, wherein the deforming provides a deformation of the mesh model to converge to the segmented object model, such as a segmented bone and/or soft tissue model, to within a threshold, which provides a transformation between the at least one object, such as bone and/or soft tissue, and the image data captured by the one or more cameras, which therefore provides an accurate model, such as a bone and/or soft tissue model that includes soft tissue and bone, which provides the position/pose of the object, such as the bone and/or soft tissue.
A24. The method of any of A1 to A23, wherein the image/image data is obtained from a plurality of cameras, wherein each camera of the plurality of cameras provides a respective image stream such that a corresponding plurality of image streams are provided by the plurality of cameras, wherein the generating the point cloud further comprises preprocessing the plurality of image streams by one or more of a collection of edge computing device(s) to produce and output preprocessed data.
A25. The method of A24, wherein the preprocessing comprises at least one selected from the group consisting of: (i) cropping, (ii) color filtering, (iii) segmenting (as at least some of the “segmenting the generated point cloud” or other segmenting), (iv) bounding box processing that locates/identifies objects within a region (bounding box) of an image to define spatial extent of an object for feature extraction/further analysis on the object within that region; and (v) registration.
A26. The method of any of A24 to A25, wherein the collection of edge computing device(s) comprises a merge component that merges preprocessed image streams (as intermediate preprocessed data) to produce the output preprocessed data.
A27. The method of any of A24 to A26, wherein the preprocessing generates the point cloud and the generated point cloud is output as part of the output preprocessed data.
A28. The method of any of A24 to A27, wherein the preprocessing performs at least some of the segmenting.
A29. The method of any of A24 to A28, wherein the preprocessed data is output to an application, the application to perform a collection of tasks in connection with a guided robotic surgery, the collection of tasks reliant on the plurality of image streams having been preprocessed into the preprocessed data, wherein the collection of the edge computing device(s) are not dedicated/configured to and/or do not perform the collection of tasks that the application is to perform.
A30. The method of any of A1 to A29, further comprising rigidly fixating the at least one object to at least one other object to minimize movement of the at least one object.
A31. The method of A30, wherein the rigidly fixating minimizes movement of the at least one object in space to 1 millimeter or less.
A32. A computer-implemented method for robotic surgery, the method comprising: navigating/controlling a robot during a navigated robotic surgery based on markerless tracking of at least one object.
A33. The method of A32, wherein the navigating/controlling comprises maneuvering the robot and/or controlling the robot during performance of a cut made to the at least one object.
A34. A computer-implemented method for modeling patient anatomy in a current position and pose, including registering bone that is obscured by soft tissue, the method comprising: generating a point cloud of the patient anatomy; segmenting the point cloud to identify visible bone surface regions of the patient anatomy and visible soft tissue surface regions of the patient anatomy; registering a reference bone model for the patient to the identified visible bone surface regions, wherein the registering provides an initial patient anatomy model having an estimation of a pose of bone portions [estimated bone regions] of the patient anatomy; augmenting the initial patient anatomy model with soft tissue bodies, each comprising a respective volume and respective surface, based on the estimation of the pose of the bone portions [estimated bone regions] of the patient anatomy, wherein the augmenting provides a full patient anatomy model having (i) the soft tissue bodies representing soft tissue portions of the patient anatomy and (ii) elements representing the bone portions of the patient anatomy; and registering the full patient anatomy model to the segmented point cloud such that the full patient anatomy model accurately reflects a current position and pose of the patient anatomy.
A35. The method of A34, wherein the generating uses laser-based depth ranging/detection.
A36. The method of A35, wherein the generating uses one or more cameras that incorporate light detection and ranging (LIDAR) technology for the laser-based depth ranging/detection.
A37. The method of A34, wherein the segmenting also identifies anatomical landmarks.
A38. The method of A37, wherein the landmarks comprise most distal points of articulating cartilage surfaces.
A39. The method of A34, wherein the segmenting uses a machine-learning model to perform the segmenting.
A40. The method of A34, further comprising receiving user-provided indication(s) of soft tissue and/or bone regions, wherein the segmenting uses the user-provided indication(s).
A41. The method of A34, wherein the reference bone model is a reference model of the bone portions derived from (i) a patient-specific CT scan of the patient or (ii) another anatomical dataset, the another anatomical dataset being a generalized model of the anatomy.
A42. The method of A34, wherein the augmenting provides the soft tissue bodies on a distal femur bone surface of the full patient anatomy model and proximal tibial bone surface of the full patient anatomy model.
A43. The method of A34, wherein the augmenting provides the soft tissue bodies such that articulation of femur cartilage grossly matches an articulating distal surface of femur bone represented in the full patient anatomy model, and a flat surface of tibia cartilage grossly matches a flat surface of a proximal surface of tibia bone represented in the full patient anatomy model.
A44. The method of A34, wherein the augmenting comprises deforming elements of the full patient anatomy model, the elements comprising at least the soft tissue bodies.
A45. The method of A44, wherein the deforming uses assumed constraints on properties of the soft tissue bodies, including constraints as to soft tissue thickness/depth in one or more soft tissue regions of the patient anatomy.
A46. The method of A45, wherein the assumed constraints are based on prior known anatomical approximation of the soft tissue thickness.
A47. The method of A44, wherein the deforming emphasizes deforming the soft tissue bodies over deforming the elements representing the bone portions of the patient anatomy.
A48. The method of A44, wherein the deforming uses (i) first constraints on deforming the soft tissue bodies anatomy and (ii) second constraints on deforming the elements representing the bone portions of the patient anatomy, the first constraints and second constraints provided as weights for the deforming.
A49. The method of A34, wherein the registering the full patient anatomy model to the segmented point cloud uses a multi-hypothesis registration method.
A50. The method A34, wherein the bone portions are estimated bone regions.
A51. A computer system comprising: a memory; and a processing circuit in communication with the memory, wherein the computer system is configured to perform a method of any of A1-A50.
A52. A computer program product comprising: a computer readable storage medium readable by a processing circuit and storing instructions for execution by the processing circuit for performing a method of any of claims A1-A50.

What is claimed is:

1. A computer-implemented method for modeling patient anatomy in a current position and pose, including registering bone that is obscured by soft tissue, the method comprising:
generating a point cloud of the patient anatomy;
segmenting the point cloud to identify visible bone surface regions of the patient anatomy and visible soft tissue surface regions of the patient anatomy;
registering a reference bone model for the patient to the identified visible bone surface regions, wherein the registering provides an initial patient anatomy model having an estimation of a pose of bone portions of the patient anatomy;
augmenting the initial patient anatomy model with soft tissue bodies, each comprising a respective volume and respective surface, based on the estimation of the pose of the bone portions of the patient anatomy, wherein the augmenting provides a full patient anatomy model having (i) the soft tissue bodies representing soft tissue portions of the patient anatomy and (ii) elements representing the bone portions of the patient anatomy; and
registering the full patient anatomy model to the segmented point cloud such that the full patient anatomy model accurately reflects a current position and pose of the patient anatomy.

2. The method of claim 1, wherein the segmenting also identifies anatomical landmarks.

3. The method of claim 2, wherein the landmarks comprise most distal points of articulating cartilage surfaces.

4. The method of claim 1, further comprising receiving user-provided indications of one or more of soft tissue or bone regions, wherein the segmenting uses the user-provided indications.

5. The method of claim 1, wherein the augmenting provides the soft tissue bodies such that articulation of femur cartilage grossly matches an articulating distal surface of femur bone represented in the full patient anatomy model, and a flat surface of tibia cartilage grossly matches a flat surface of a proximal surface of tibia bone represented in the full patient anatomy model.

6. The method of claim 1, wherein the augmenting comprises deforming elements of the full patient anatomy model, the elements comprising at least the soft tissue bodies.

7. The method of claim 6, wherein the deforming uses assumed constraints on properties of the soft tissue bodies, including constraints as to one or more of soft tissue thickness or depth in one or more soft tissue regions of the patient anatomy.

8. The method of claim 7, wherein the assumed constraints are based on prior known anatomical approximation of the soft tissue thickness.

9. The method of claim 6, wherein the deforming emphasizes deforming the soft tissue bodies over deforming the elements representing the bone portions of the patient anatomy.

10. The method of claim 6, wherein the deforming uses (i) first constraints on deforming the soft tissue bodies anatomy and (ii) second constraints on deforming the elements representing the bone portions of the patient anatomy, the first constraints and second constraints provided as weights for the deforming.

11. The method of claim 1, wherein the registering the full patient anatomy model to the segmented point cloud uses a multi-hypothesis registration method.

12. A computer system comprising:
a memory; and
a processor in communication with the memory, wherein the computer system is configured to perform a method for modeling patient anatomy in a current position and pose, including registering bone that is obscured by soft tissue, the method comprising:
generating a point cloud of the patient anatomy;
segmenting the point cloud to identify visible bone surface regions of the patient anatomy and visible soft tissue surface regions of the patient anatomy;
registering a reference bone model for the patient to the identified visible bone surface regions, wherein the registering provides an initial patient anatomy model having an estimation of a pose of bone portions of the patient anatomy;
augmenting the initial patient anatomy model with soft tissue bodies, each comprising a respective volume and respective surface, based on the estimation of the pose of the bone portions of the patient anatomy, wherein the augmenting provides a full patient anatomy model having (i) the soft tissue bodies representing soft tissue portions of the patient anatomy and (ii) elements representing the bone portions of the patient anatomy; and
registering the full patient anatomy model to the segmented point cloud such that the full patient anatomy model accurately reflects a current position and pose of the patient anatomy.

13. The computer system of claim 12, wherein the segmenting also identifies anatomical landmarks.

14. The computer system of claim 12, wherein the method further comprises receiving user-provided indications of one or more of soft tissue or bone regions, wherein the segmenting uses the user-provided indications.

15. The computer system of claim 12, wherein the augmenting provides the soft tissue bodies such that articulation of femur cartilage grossly matches an articulating distal surface of femur bone represented in the full patient anatomy model, and a flat surface of tibia cartilage grossly matches a flat surface of a proximal surface of tibia bone represented in the full patient anatomy model.

16. The computer system of claim 12, wherein the augmenting comprises deforming elements of the full patient anatomy model, the elements comprising at least the soft tissue bodies.

17. The computer system of claim 16, wherein the deforming uses assumed constraints on properties of the soft tissue bodies, including constraints as to one or more of soft tissue thickness or depth in one or more soft tissue regions of the patient anatomy.

18. The computer system of claim 16, wherein the deforming emphasizes deforming the soft tissue bodies over deforming the elements representing the bone portions of the patient anatomy.

19. The computer system of claim 16, wherein the deforming uses (i) first constraints on deforming the soft tissue bodies anatomy and (ii) second constraints on deforming the elements representing the bone portions of the patient anatomy, the first constraints and second constraints provided as weights for the deforming.

20. The computer system of claim 12, wherein the registering the full patient anatomy model to the segmented point cloud uses a multi-hypothesis registration method.
